(19) United States
(12) Patent Application Publication
(10) Pub. No.: US 2022/0018835 A1
(43) Pub. Date: Jan. 20, 2022

(54) USE OF CD26 AND CD39 AS NEW PHENOTYPIC MARKERS FOR ASSESSING MATURATION OF FOXP3+ T CELLS AND USES THEREOF FOR DIAGNOSTIC PURPOSES

(71) Applicants: INSERM (Institut National de la Santé et de la Recherche Médicale, Paris (FR); UNIVERSITE DE PARIS, Paris (FR); MEDECINE ET INNOVATION, Paris (FR); FONDS DE LA RECHERCHE SCIENTIFIQUE - FNRS, BRUXELLES (BE)

(72) Inventors: Daniel ZAGURY, Paris (FR); Hélène LE BUANEC, Paris (FR); Armand BENSUSSAN, Paris (FR); Charles CASSIUS, Paris Cedex 10 (FR)

(21) Appl. No.: 17/311,509

(22) PCT Filed: Dec. 6, 2019
(86) PCT No.: PCT/EP2019/083928

§ 371 Date: Jun. 7, 2021

(30) Foreign Application Priority Data
Dec. 7, 2018 (EP) 18306641.4

(51) Int. Cl. G01N 33/564; C12N 5/0783; G01N 33/68

(52) U.S. Cl. CPC G01N 33/564; C12N 5/0637; G01N 33/6893; G01N 2800/50; C12N 2501/15; C12N 2501/2302; G01N 2800/24; C12N 2501/02

(57) ABSTRACT

Among regulatory T cells, natural regulatory T cells (nTregs) ensure the control of self-tolerance and are currently tested in clinical trials in autoimmune diseases and allogeneic hematopoietic stem cell transplantation. Here the inventors show that based on CD39/CD26 markers, the human nTreg population is comprised of 5 major cell subsets each representing a distinct state of maturation. Phenotypic and genetic characteristics of the subsets illustrate the structural parental maturation between subsets which further correlates with expression of regulatory factors. Importantly, the inventors also show that blood nTreg CD39/CD26 profile, remaining constant over a 2year period in healthy persons but varying between individuals, represents a novel biomarker for monitoring chronic diseases, as illustrated in their preliminary study on AI (dermatomyositis, rheumatoid arthritis and leukemias). Accordingly, the present invention relates to the use of CD26 and CD39 as phenotypic markers for assessing maturation of natural Treg cells.

### FIELD OF THE INVENTION

The present invention relates to new phenotypic markers for assessing maturation of Foxp3+ T cells and their uses thereof for diagnostic purposes.

### BACKGROUND OF THE INVENTION

Immune suppressive T cells contribute to multiple functions such as tolerance to self-antigens (Ags) or to foreign Ags generated during pregnancy and food digestion (1, 2). These vital suppressive activities are carried out by different CD4 and CD8 regulatory T cell types (1-5) through an apparent redundancy of regulatory mechanisms (6). Dysfunction of regulatory T cells leads to severe chronic pathologies, including autoimmune diseases (AID) (1), viral infections (7), cancer (8), allergy (9) and graft versus host disease (GVHD) (10).

Among suppressive T cell populations, three critical conventional regulatory T cell types are structurally and functionally well identified: 1) type 1 T regulatory cells (Tr1) which are characterized by their release of IL-10 (IL10 Tr1), thus controlling over-inflammation/danger during adaptive immune reactions (IR) (11). Given that these cells originate from different activated T cell types, including Th2, Th1, Th17, Treg and CD8 T cells, IL-10 Tr1 cells should not be considered as a specific T cell lineage but rather as activated T cells skewing their functional differentiation under overinflammation/danger to a reprogrammed suppressive Tr1 function, preventing immunopathogenesis (12, 13). 2) The suppressive Ag specific HLA-E restricted CD8 T cells (TCD8sup), which lyse by cell contact activated CD4+ T cell targets expressing the stimulating peptide Ag-HLA-E complex (14). The suppressive CD8 T cells express CD44, CD122, KIR and the transcription factor Helios (15). 3) CD4 nTreg cells introduced by Sakaguchi, and initially identified by their thymic developmental origin, their CD25 high phenotype and following TCR stimulation, their cell-cell contact-mediated suppressive activity toward activated immune cells, including autoreactive ones, thus implicating them in the control of self-tolerance (1). Since then, numerous reports have extended our basic knowledge on these cells. These reports showed that human nTregs are characterized by the high MFI expression of the master FOXP3 transcript (16) , which may also be expressed by activated CD4+ (17) and CD8+ (18) T cells. They further showed that nTregs not only produce the anti-inflammatory IL-10 (10), but also its proinflammatory IL-17 counterpart (19, 20). Remarkably, these FOXP3 lineage Tregs can be induced in culture by appropriate stimulation of naïve CD45RA⁺CD4⁺ T cells (TH0) in the presence of IL-2 and TGF-β (iTregs) (21, 22). Most importantly the regulatory function of these cells in the control of self-tolerance prompted experimentations and clinical trials, based on the transfer of nTreg populations to treat GVHD or autoimmune pathologies (23). The results concerning the adoptive transfer of crude nTreg preparations although encouraging (24), are as yet not fully satisfactory and treatment failures were attributed to inflammatory complications triggered by the capacity of nTregs to behave as proinflammatory Th17 cells (19, 20). The need to significantly improve nTreg preparations for a more effective therapeutic benefit was further confirmed by a set of experimental data, which pointed to a functional heterogeneity of the FOXP3 Treg populations currently administered in adoptive IT. For instance, in a study on nTregs carried out in healthy and lupus-affected individuals, we reported that the suppressive activities of human blood nTregs, as tested by the current suppressive assay (i.e. following stimulation by polyclonal anti-CD3 Abs), varied according to the stimulatory environmental conditions. Precisely in a culture medium corresponding to an in vivo quiescent stromal tissue (steady state) nTreg cells effectively inhibited activation and proliferation of their co-cultured autologous cell targets by a cell-cell contact-mediated, and not by a humoral mechanism, while in culture conditions mimicking the in vivo inflammatory adaptive reactions to pathogenic antigens (13), the cell-contact mediated suppressive effect was inhibited, but nTregs unexpectedly released IL-10.

### SUMMARY OF THE INVENTION

The present invention relates to new phenotypic markers for assessing maturation of Foxp3+ T cells and their uses thereof for diagnostic purposes. In particular, the present invention is defined by the claims.

### DETAILED DESCRIPTION OF THE INVENTION

Among regulatory T cells, natural regulatory T cells (nTregs) ensure the control of self-tolerance and are currently tested in clinical trials in autoimmune diseases and allogeneic hematopoietic stem cell transplantation. Here the inventors show that based on CD39/CD26 markers, the human nTreg population is comprised of 5 major cell subsets each representing a distinct state of maturation. They found that in vitro microenvironmental factors including IL-2, TGF-β and PGE2 directed the conversion from naïve precursor to immature memory subsets and from immature to mature cells, the latest being a no return stage. Phenotypic and genetic characteristics of the subsets illustrate the structural parental maturation between subsets which further correlates with expression of regulatory factors. NTreg subsets functions include blockade of autoreactive immune cells, activation of TCR-stimulating DC tolerization, TH-17 like and IL-10 TR1 like activity and expression of these functions is conditioned by both their stage of maturation and the quiescent, pro-inflammatory or over-inflammatory microenvironmental context of their TCR stimulation, respectively. Importantly, the inventors also show that blood nTreg CD39/CD26 profile, remaining constant over a 2 year period in healthy persons but varying between individuals, represents a novel biomarker for monitoring chronic inflammatory diseases, as illustrated in their preliminary study on AI (dermatomyositis, rheumatoid arthritis and leukemias).

Main Definitions:

As used herein, the term “T cell” has its general meaning in the art and refers to a type of lymphocytes that play an important role in cell-mediated immunity and are distinguished from other lymphocytes, such as B cells, by the presence of a T-cell receptor (TCR) on the cell surface. In particular, T cells are characterised by the expression of CD3. The term “CD3” refers to the protein complex associated with the T cell receptor is composed of four distinct chains. In mammals, the complex contains a CD3γ chain, a CD3δ chain, and two CD3ϵ chains. These chains associate with the TCR and the ζ-chain (zeta-chain) to generate an activation signal in T lymphocytes. The TCR, ζ-chain, and CD3 molecules together constitute the TCR complex. In particular, T cells are characterized by the expression of CD4 or CD8 and thus be classified as CD4+ T cells and CD8+ cells. As used herein, the term “CD4” has its general meaning in the art and refers to the T-cell surface glycoprotein CD4. CD4 is a co-receptor of the T cell receptor (TCR) and assists the latter in communicating with antigen-presenting cells. The TCR complex and CD4 each bind to distinct regions of the antigen-presenting MHCII molecule—α1/β1 and β2, respectively.

As used herein, the term “CD8” has its general meaning in the art and refers to the T c ell surface glycoprotein CD8. In particular, CD8 is a transmembrane glycoprotein that serves as a co-receptor for the T cell receptor (TCR). Like the TCR, CD8 binds to a major histocompatibility complex (MHC) molecule, but is specific for the class I MHC protein.

As used herein, the term “CD8+ T cell” has its general meaning in the art and refers to a subset of T cells which express CD8 on their surface. They are MHC class I-restricted, and function as cytotoxic T cells. “CD8+ T cells” are also called cytotoxic T lymphocytes (CTL), T-killer cells, cytolytic T cells, or killer T cells. CD8 antigens are members of the immunoglobulin supergene family and are associative recognition elements in major histocompatibility complex class I-restricted interactions.

As used herein, the term “CD4+ T cells” has its general meaning in the art and refers to a subset of T cells which express CD4 on their surface. CD4+ T cells are T helper cells, which either orchestrate the activation of macrophages and CD8+ T cells (Th-1 cells), the production of antibodies by B cells (Th-2 cells) or which have been thought to play an essential role in autoimmune diseases (Th-17 cells).

As used herein, “Treg” or “regulatory T cells” refers to cells functionally committed, i.e. capable of suppressive activity (i.e. inhibiting proliferation of conventional T cells), either by cell-cell contact or by MLR suppression (Mixed Lymphocytes Reaction).

As used herein, the term “nTregs” or “natural regulatory T cells” has its general meaning in the art and refers to regulatory T cells characterized by their thymic development origin, their CD4⁺CD25⁺CD127⁻Foxp3+ phenotype and their TSDR (Treg specific demethylated region). nTregs are thus characterized by the expression of Foxp3 and CD4.

As used, the term “Foxp3” has its general meaning in the art and refers to a transcriptional regulator which is crucial for the development and inhibitory function of Treg. Foxp3 plays an essential role in maintaining homeostasis of the immune system by allowing the acquisition of full suppressive function and stability of the Treg lineage, and by directly modulating the expansion and function of conventional T-cells. Foxp3 can act either as a transcriptional repressor or a transcriptional activator depending on its interactions with other transcription factors, histone acetylases and deacetylases. Foxp3 inhibits cytokine production and T-cell effector function by repressing the activity of two key transcription factors, RELA and NFATC2. The factor also mediates transcriptional repression of IL2 via its association with histone acetylase KATS and histone deacetylase HDAC7. Foxp3 can activate the expression of TNFRSF18, IL2RA and CTLA4 and repress the expression of IL2 and IFNG via its association with transcription factor RUNX1. Foxp3 inhibits the differentiation of IL17 producing helper T-cells (Th17) by antagonizing RORC function, leading to down-regulation of IL17 expression, favoring Treg development. An exemplary human amino acid sequence for Foxp3 is represented by SEQ ID NO:1.

>sp|Q9BZS1|FOXP3_HUMAN Forkhead box protein
P3 OS = Homo sapiens OX = 9606 GN = FOXP3 PE = 1
SV = 1
SEQ ID NO: 1
MPNPRPGKPSAPSLALGPSPGASPSWRAAPKASDLLGARGPGGTFQGRDLR
GGAHASSSSLNPMPPSQLQLPTLPLVMVAPSGARLGPLPHLQALLQDRPHE
MHQLSTVDAHARTPVLQVHPLESPAMISLTPPTTATGVESLKARPGLPPGI
NVASLEWVSREPALLCTFPNPSAPRKDSTLSAVPQSSYPLLANGVCKWPGC
EKVFEEPEDFLKHCQADHLLDEKGRAQCLLQREMVQSLEQQLVLEKEKLSA
MQAHLAGKMALTKASSVASSDKGSCCIVAAGSQGPVVPAWSGPREAPDSLF
AVRRHLWGSHGNSTFPEFLHNMDYFKFHNMRPPFTYATLIRWAILEAPEKQ
RTLNEIYHWFTRMFAFFRNHPATWKNAIRHNLSLHKCFVRVESEKGAVWTV
DELEFRKKRSQRPSRCSNPTPGP

As used herein, the term “CD25” has its general meaning in the art and refers to the alpha chain of the human interleukin-2 receptor. The interleukin 2 (IL2) receptor alpha (IL2RA) and beta (IL2RB) chains, together with the common gamma chain (IL2RG), constitute the high-affinity IL2 receptor. Homodimeric alpha chains (IL2RA) result in low-affinity receptor, while homodimeric beta (IL2RB) chains produce a medium-affinity receptor. An exemplary human amino acid sequence for CD25 is represented by SEQ ID NO:2.

>sp|P01589|IL2RA_HUMAN Interleukin-2 receptor
subunit alpha OS = Homo sapiens OX = 9606
GN = IL2RA PE = 1 SV = 1
SEQ ID NO: 2
MDSYLLMWGLLTFIMVPGCQAELCDDDPPEIPHATFKAMAYKEGTMLNCEC
KRGFRRIKSGSLYMLCTGNSSHSSWDNQCQCTSSATRNTTKQVTPQPEEQK
ERKTTEMQSPMQPVDQASLPGHCREPPPWENEATERIYHFVVGQMVYYQCV
QGYRALHRGPAESVCKMTHGKTRWTQPQLICTGEMETSQFPGEEKPQASPE
GRPESETSCLVTTTDFQIQTEMAATMETSIFTTEYQVAVAGCVFLLISVLL
LSGLTWQRRQRKSRRTI

As used herein, the term “CD26” has its general meaning in the art and refers to the cell surface glycoprotein receptor involved in the costimulatory signal essential for T-cell receptor (TCR)-mediated T-cell activation. CD26 indeed acts as a positive regulator of T-cell coactivation, by binding at least adenosine deaminase (ADA) and thus regulates lymphocyte-epithelial cell adhesion. An exemplary human amino acid sequence for CD26 is represented by SEQ ID NO:3.

>sp|P27487|DPP4_HUMAN Dipeptidyl peptidase 4
OS = Homo sapiens OX = 9606 GN = DPP4 PE = 1 SV = 2
SEQ ID NO: 3
MKTPWKVLLGLLGAAALVTIITVPVVLLNKGTDDATADSRKTYTLIDYLKN
TYRLKLYSLRWISDHEYLYKQENNILVFNAEYGNSSVFLENSTFDEFGHSI
NDYSISPDGQFILLEYNYVKQWRHSYTASYDIYDLNKRQLITEERIPNNTQ
WVIWSPVGHKLAYVWNNDIYVKIEPNLPSYRITWIGKEDIIYNGITDWVYE
EEVFSAYSALWWSPNGTFLAYAQFNDTEVPLIEYSFYSDESLQYPKTVRVP
YPKAGAYNPTVKFFVVNTDSLSSVINATSIQITAPASMLIGDHYLCDVTWA
TQERISLQWLRRIQNYSVMDICDYDESSGRWNCLVARQHIEMSTIGWVGRF
RPSEPHFILDGNSFYKIISNEEGYRHICYFQIDKKDCIFITKGTWEVIGIE
ALTSDYLYYISNEYKGMPGGRNLYKIQLSDYTKVICLSCELNPERCQYYSV
SFSKEAKYYQLRCSGPGLPLYILHSSVNDKGLRVLEDNSALDKMLQNVQMP
SKKLDFIILNETKFWYQMILPPHFDKSKKYPLLLDVYAGPCSQKADTVERL
NWATYLASTENIIVASEDGRGSGYQGDKIMHAINRRLGTFEVEDQIEAARQ
FSKMGFVDNKRIAIWGWSYGGYVISMVLGSGSGVFKCGIAVAPVSRWEYYD
SVYTERYMGLPTPEDNLDHYRNSTVMSRAENFKQVEYLLIHGTADDNVHFQ
QSAQISKALVDVGVDFQAMWYTDEDHGIASSTAHQHIYTHMSHFIKQCFSL
P

As used herein, the term “CD39” has its general meaning in the art and refers to the ectonucleoside triphosphate diphosphohydrolase-1 (ENTPD1) that is an ectoenzyme that hydrolases ATP/UTP and ADP/UDP to the respective nucleosides such as AMP. An exemplary human amino acid sequence for CD39 is represented by SEQ ID NO:4.

>sp|P49961|ENTP1_HUMAN Ectonucleoside triphosphate
diphosphohydrolase 1 OS = Homo sapiens OX = 9606
GN = ENTPD1 PE = 1 SV = 1
SEQ ID NO: 4
MEDTKESNVKTFCSKNILAILGFSSIIAVIALLAVGLTQNKALPENVKYGI
VLDAGSSHTSLYIYKWPAEKENDTGVVHQVEECRVKGPGISKFVQKVNEIG
IYLTDCMERAREVIPRSQHQETPVYLGATAGMRLLRMESEELADRVLDVVE
RSLSNYPFDFQGARIITGQEEGAYGWITINYLLGKFSQKTRWFSIVPYETN
NQETFGALDLGGASTQVTFVPQNQTIESPDNALQFRLYGKDYNVYTHSFLC
YGKDQALWQKLAKDIQVASNEILRDPCFHPGYKKVVNVSDLYKTPCTKRFE
MTLPFQQFEIQGIGNYQQCHQSILELFNTSYCPYSQCAFNGIFLPPLQGDF
GAFSAFYFVMKFLNLTSEKVSQEKVTEMMKKFCAQPWEEIKTSYAGVKEKY
LSEYCFSGTYILSLLLQGYHFTADSWEHIHFIGKIQGSDAGWTLGYMLNLT
NMIPAEQPLSTPLSHSTYVFLMVLFSLVLFTVAIIGLLIFHKPSYFWKDMV

As used herein the term “CD45” has its general meaning in the art and refers to the protein tyrosine phosphatase (PTP) encoded by the PTPRC gene, which is specifically expressed in hematopoietic cells. CD45 regulates receptor signalling by direct interaction with components of the receptor complexes or by activating and dephosphorylating various Src family kinases (SFK) i.e. Lck. But it can inhibit cytokine receptor signalling by inhibiting JAK kinases or by dephosphorylating the activating residues of Src. Typically it is possible to distinguish two isoforms of CD45: CD45RA and CD45RO. The term “CD45RA” relates to the isoform in which exon 4 of the CD45 gene is not expressed. The term “CD45RO” refers to the CD45 isoform in which exons 4, 5, and 6 of the CD45 gene are not expressed. An exemplary human amino acid sequence for CD45RA is represented by SEQ ID NO:5.

>sp|P08575|PTPRC_HUMAN Receptor-type tyrosine-
protein phosphatase C OS = Homo sapiens OX = 9606
GN = PTPRC PE = 1 SV = 3
SEQ ID NO: 5
MTMYLWLKLLAFGFAFLDTEVFVTGQSPTPSPTGLTTAKMPSVPLSSDPLP
THTTAFSPASTFERENDFSETTTSLSPDNTSTQVSPDSLDNASAFNTTGVS
SVQTPHLPTHADSQTPSAGTDTQTFSGSAANAKLNPTPGSNAISDVPGERS
TASTFPTDPVSPLTTTLSLAHHSSAALRARTSNTTITANTSDAYLNASETT
TLSPSGSAVISTTTIATTPSKPTCDEKYANITVDYLYNKETKLFTAKLNVN
ENVECGNNTCTNNEVHNLTECKNASVSISHNSCTAPDKTLILDVPPGVEKF
QLHDCTQVEKADTTICLKWKNIETFTCDTQNITYRFQCGNMIEDNKEIKLE
NLEPEHEYKCDSEILYNNHKFTNASKIIKTDFGSPGEPQIIFCRSEAAHQG
VITWNPPQRSFHNFTLCYIKETEKDCLNLDKNLIKYDLQNLKPYTKYVLSL
HAYITAKVQRNGSAAMCHFTTKSAPPSQVWNMTVSMTSDNSMHVKCRPPRD
RNGPHERYHLEVEAGNTLVRNESHKNCDFRVKDLQYSTDYTFKAYFHNGDY
PGEPFILHHSTSYNSKALIAFLAFLIIVTSIALLVVLYKIYDLHKKRSCNL
DEQQELVERDDEKQLMNVEPIHADILLETYKRKIADEGRLFLAEFQSIPRV
FSKFPIKEARKPFNQNKNRYVDILPYDYNRVELSEINGDAGSNYINASYID
GFKEPRKYIAAQGPRDETVDDEWRMIWEQKATVIVMVTRCEEGNRNKCAEY
WPSMEEGTRAFGDVVVKINQHKRCPDYIIQKLNIVNKKEKATGREVTHIQF
TSWPDHGVPEDPHLLLKLRRRVNAFSNFFSGPIVVHCSAGVGRTGTYIGID
AMLEGLEAENKVDVYGYVVKLRRQRCLMVQVEAQYILIHQALVEYNQFGET
EVNLSELHPYLHNMKKRDPPSEPSPLEAEFQRLPSYRSWRTQHIGNQEENK
SKNRNSNVIPYDYNRVPLKHELEMSKESEHDSDESSDDDSDSEEPSKYINA
SFIMSYWKPEVMIAAQGPLKETIGDFWQMIFQRKVKVIVMLTELKHGDQEI
CAQYWGEGKQTYGDIEVDLKDTDKSSTYTLRVFELRHSKRKDSRTVYQYQY
TNWSVEQLPAEPKELISMIQVVKQKLPQKNSSEGNKHHKSTPLLIHCRDGS
QQTGIFCALLNLLESAETEEVVDIFQVVKALRKARPGMVSTFEQYQFLYDV
IASTYPAQNGQVKKNNHQEDKIEFDNEVDKVKQDANCVNPLGAPEKLPEAK
EQAEGSEPTSGTEGPEHSVNGPASPALNQGS

As used herein, the term “CD127” has its general meaning in the art and refers to the interleukin-7 receptor subunit alpha. An exemplary human amino acid sequence for CD127 is represented by SEQ ID NO:6.

>sp|P16871|IL7RA_HUMAN Interleukin-7 receptor
subunit alpha OS = Homo sapiens OX = 9606 GN = IL7R
PE = 1 SV = 2
SEQ ID NO: 6
MTILGTIFGMVFSLLQVVSGESGYAQNGDLEDAELDDYSFSCYSQLEVNGS
QHSLICAFEDPDVNTINLEFEICGALVEVKCLNFRKLQEIYFIETKKFLLI
GKSNICVKVGEKSLICKKIDLITIVKPEAPFDLSVIYREGANDFVVIFNIS
HLQKKYVKVLMHDVAYRQEKDENKWTHVNLSSTKLILLQRKLQPAAMYEIK
VRSIPDHYFKGFWSEWSPSYYFRIPEINNSSGEMDPILLTISILSFFSVAL
LVILACVLWKKRIKPIVWPSLPDHKKTLEHLCKKPRKNLNVSFNPESFLDC
QIHRVDDIQARDEVEGFLQDTFPQQLEESEKQRLGGDVQSPNCPSEDVVIT
PESFGRDSSLICLAGNVSACDAPILSSSRSLDCRESGKNGPHVYQDLLLSL
GTINSTLPPPFSLQSGILTLNPVAQGQPILTSLGSNQEEAYVTMSSFYQNQ

As used herein, the term “expression” may refer alternatively to the transcription of a molecule (i.e. expression of the mRNA) or to the translation (i.e. expression of the protein) of a molecule. In some embodiments, detecting the expression may correspond to an intracellular detection. In some embodiments, detecting the expression may correspond to a surface detection, i.e. to the detection of molecule expressed at the cell surface. In some embodiments, detecting the expression may correspond to an extracellular detection, i.e. to the detection of secretion. In some embodiments, detecting the expression may correspond to intracellular, surface and/or extracellular detections.

As used herein, the terms “expressing (or +)” and “not expressing (or −)” are well known in the art and refer to the expression level of the phenotypic marker of interest, in that the expression level of the phenotypic marker corresponding to “+” is high or intermediate, also referred as “+/−”. The phenotypic marker corresponding to “−” is a null expression level of the phenotypic marker or also refers to less than 10% of a cell population expressing the said phenotypic marker.

As used herein, the term “isolated population” refers to a cell population that is removed from its natural environment (such as the peripheral blood or a tissue) and that is isolated, purified or separated, and is at least about 75% free, 80% free, 85% free and preferably about 90%, 95%, 96%, 97%, 98%, 99% free, from other cells with which it is naturally present, but which lack the cell surface markers based on which the cells were isolated.

As used herein, the term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein. By “antibody” or “antibody molecule”, it is intended herein not only whole immunoglobulin molecules but also fragments thereof, such as Fab, F(ab′)2, Fv and other fragments thereof. Similarly, the term antibody includes genetically engineered derivatives of antibodies such as single chain Fv molecules (scFv) and domain antibodies (dAbs). The term “monoclonal antibody” is used herein to encompass any isolated Ab′s such as conventional monoclonal antibody hybridomas, but also to encompass isolated monospecific antibodies produced by any cell, such as for example a sample of identical human immunoglobulins expressed in a mammalian cell line. Suitable monoclonal antibodies which are reactive as described herein may be prepared by known techniques, for example those disclosed in “Monoclonal Antibodies; A manual of techniques”, H Zola (CRC Press, 1988) and in “Monoclonal Hybridoma Antibodies: Techniques and Application”, S G R Hurrell (CRC Press, 1982).

As used herein, the terms “label” or “tag” refer to a composition capable of producing a detectable signal indicative of the presence of a target, such as, the presence of a specific phenotypic marker in a biological sample.

As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors.

As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest.

As used herein, “subject or patient” refers to a mammal, preferably a human. In the present invention, the terms subject and patient may be used with the same meaning. In some embodiments, the subject is awaiting the receipt of, or is receiving medical care or was/is/will be the object of a medical procedure, or is monitored for the development of a disease. In some embodiments, the subject is an adult (for example a subject above the age of 18). In some embodiments, the subject is a child (for example a subject below the age of 18). In some embodiments, the subject is an elderly human (for example a subject above the age of 60). In some embodiments, the subject is a male. In some embodiments, the subject is a female.

As used herein, the term “sample” refers to a biological sample obtained for the purpose of in vitro evaluation. In the methods of the invention, the biological samples include, but are not limited to, body fluid samples (such as blood and blood serum), plasma, synovial fluid, bronchoalveolar lavage fluid, sputum, lymph, ascetic fluids, peritoneal fluid, cerebrospinal fluid, pleural fluid, pericardial fluid, and alveolar macrophages, tissue lysates, solid tissue, diseased tissues samples (e.g., tumorous or cancerous tissues). In some aspects, the tissue sample may be obtained by methods known in the art including, without limitation, tissue biopsy, fine needle aspiration sample, surgically resected tissue, or histological preparations of a biological sample. In some aspect, biological samples to be used in the methods according to the invention may be blood samples (e.g. whole blood sample or PBMC sample). A blood sample may be obtained by methods known in the art including venipuncture or a finger stick. Serum and plasma samples may be obtained by centrifugation methods known in the art. The sample may be diluted with a suitable buffer before conducting the assay. As used herein, the term “PBMC” or “peripheral blood mononuclear cells” or “unfractionated PBMC”, as used herein, refers to whole PBMC, i.e. to a population of white blood cells having a round nucleus, which has not been enriched for a given sub-population. Cord blood mononuclear cells are further included in this definition. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer which will preferentially lyse red blood cells. Such procedures are known to the expert in the art.

As used herein, the term “impaired immune response” refers to a state in which a subject does not have an appropriate immune response, e.g., to cancer, vaccination, pathogen infection, among others. In some embodiments, a subject having an impaired immune response is predicted not to get protective antibody titer levels following prophylactic vaccination, or in which a subject does not have a decrease in disease burden after therapeutic vaccination. A subject can also have an impaired immune response if the subject is a member of a population known to have decreased immune function or that has a history of decreased immune function such as the elderly, subjects undergoing chemotherapy treatment, asplenic subjects, immunocompromised subjects, or subjects having HIV/AIDS.

As used herein, the term “immunosenescence” refers to a decrease in immune function resulting in impaired immune response, e.g., to cancer, vaccination, infectious pathogens, among others. It involves both the host's capacity to respond to infections and the development of long-term immune memory, especially by vaccination. This immune deficiency is ubiquitous and found in both long- and short-lived species as a function of their age relative to life expectancy rather than chronological time. It is considered a major contributory factor to the increased frequency of morbidity and mortality among the elderly. Immunosenescence is not a random deteriorative phenomenon, rather it appears to inversely repeat an evolutionary pattern and most of the parameters affected by immunosenescence appear to be under genetic control. Immunosenescence can also be sometimes envisaged as the result of the continuous challenge of the unavoidable exposure to a variety of antigens such as viruses and bacteria. Immunosenescence is a multifactorial condition leading to many pathologically significant health problems, e.g., in the aged population.

As used herein, the term “autoimmune inflammatory disease” has its general meaning in the art and include arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, juvenile-onset rheumatoid arthritis, osteoarthritis, arthritis chronica progrediente, arthritis deformans, polyarthritis chronica primaria, reactive arthritis, ankylosing spondylitis, inflammatory hyperproliferative skin diseases, psoriasis such as plaque psoriasis, gutatte psoriasis, pustular psoriasis, psoriasis of the nails, dermatitis including contact dermatitis, chronic contact dermatitis, allergic dermatitis, allergic contact dermatitis, dermatitis herpetiformis, atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally pemphigus vulgaris, pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillo sis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to anti spermatozoan antibodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis. The term also includes autoimmune inflammatory disease secondary to therapeutic treatment, in particular a treatment with an immune checkpoint inhibitor. Typically the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies. Autoimmune inflammatory disease also include graft-related diseases, in particular, graft versus host disease (GVDH) and Host-Versus-Graft-Disease (HVGD). Typically GVHD is associated with bone marrow transplantation, and immune disorders resulting from or associated with rejection of organ, tissue, or cell graft transplantation (e.g., tissue or cell allografts or xenografts), including, e.g., grafts of skin, muscle, neurons, islets, organs, parenchymal cells of the liver, etc.

As used herein the term “infectious disease” includes any infection caused by viruses, bacteria, protozoa, molds or fungi. In some embodiments, the viral infection comprises infection by one or more viruses selected from the group consisting of Arenaviridae, Astroviridae, Birnaviridae, Bromoviridae, Bunyaviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, Tymoviridae, Hepadnaviridae, Herpesviridae, Paramyxoviridae or Papillomaviridae viruses. Relevant taxonomic families of RNA viruses include, without limitation, Astroviridae, Birnaviridae, Bromoviridae, Caliciviridae, Closteroviridae, Comoviridae, Cystoviridae, Flaviviridae, Flexiviridae, Hepevirus, Leviviridae, Luteoviridae, Mononegavirales, Mosaic Viruses, Nidovirales, Nodaviridae, Orthomyxoviridae, Picobirnavirus, Picornaviridae, Potyviridae, Reoviridae, Retroviridae, Sequiviridae, Tenuivirus, Togaviridae, Tombusviridae, Totiviridae, and Tymoviridae viruses. In some embodiments, the viral infection comprises infection by one or more viruses selected from the group consisting of adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe, pachindae viruses, adenovirus, Dengue fever, influenza A and influenza B (including human, avian, and swine), junin, measles, parainfluenza, Pichinde, punta toro, respiratory syncytial, rhinovirus, Rift Valley Fever, severe acute respiratory syndrome (SARS), Tacaribe, Venezuelan equine encephalitis, West Nile and yellow fever viruses, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and Kyasanur forest disease. Bacterial infections that can be treated according to this invention include, but are not limited to, infections caused by the following: Staphylococcus; Streptococcus, including S. pyogenes; Enterococcl; Bacillus, including Bacillus anthracis, and Lactobacillus; Listeria; Corynebacterium diphtherias; Gardnerella including G. vaginalis; Nocardia; Streptomyces; Thermoactinomyces vulgaris; Treponerna; Camplyobacter, Pseudomonas including aeruginosa; Legionella; Neisseria including N. gonorrhoeae and N. meningitides; Flavobacterium including F. meningosepticum and F. odoraturn; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachornatis; Mycobacterium including M. tuberculosis, M. intracellulare, M. folluiturn, M. laprae, M. avium, M. bovis, M. africanum, M. kansasii, M. intracellulare, and M. lepraernurium; and Nocardia. Protozoa infections that may be treated according to this invention include, but are not limited to, infections caused by leishmania, kokzidioa, and trypanosoma. A complete list of infectious diseases can be found on the website of the National Center for Infectious Disease (NCID) at the Center for Disease Control (CDC) (World Wide Web (www) at cdc.gov/ncidod/diseases/), which list is incorporated herein by reference. All of said diseases are candidates for treatment using the compositions according to the invention.

As used herein, the term “cancer” has its general meaning in the art and include solid tumors and blood-borne tumors. Example of cancers include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia (AML), Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant, Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplasia, Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, non-small cell lung cancer (NSCLC) which coexists with chronic obstructive pulmonary disease (COPD), Ocular oncology, Oligoastrocytoma, Oligodendroglioma, Oncocytoma, Optic nerve sheath, meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastema, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute, lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Vemer Morrison syndrome, Verrucous carcinoma, Visual Pathway Glioma, Vulvar Cancer, Waldenstrom's macroglobulinemia, Warthin's tumor, Wilms' tumor, or any combination thereof.

As used herein, the term “risk” in the context of the present invention, relates to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion, thus diagnosing and defining the risk spectrum of a category of subjects defined as being at risk of conversion. In the categorical scenario, the invention can be used to discriminate between normal and other subject cohorts at higher risk. In some embodiments, the present invention may be used so as to discriminate those at risk from normal.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, “therapeutic response” refers to a therapeutic benefit induced by the treatment in a subject. A therapeutic response may include the fact of (1) delaying or preventing the onset of the disease to be treated; (2) slowing down or stopping the progression, aggravation, or deterioration of one or more symptoms of the disease to be treated; (3) bringing about ameliorations of the symptoms of the disease to be treated; (4) reducing the severity or incidence of the disease to be treated; or (5) curing the disease to be treated.

As used herein the term “responder” in the context of the present disclosure refers to a subject that will achieve a response with the treatment, i.e. a subject who is under remission. A “non-responder” subject includes subjects for whom the disease does not show reduction or improvement after the treatment.

As used herein, the term “relapse” refers to the return of signs and symptoms of a disease after a subject has enjoyed a remission after a treatment. Thus, if initially the target disease is alleviated or healed, or progression of the disease was halted or slowed down, and subsequently the disease or one or more characteristics of the disease resume, the subject is referred to as being “relapsed.”

Phenotypic Markers of the Present Invention:

The inventors demonstrated that the combination of activation and maturation phenotypic markers such as CD45RA and CD25 with functional phenotypic markers such as CD26 and CD39 are particularly suitable for characterising populations of Foxp3⁺ T cells. In particular, the inventors characterized the following population of Foxp3⁺ T cells:
the population of Foxp3⁺ T cells having the following phenotype: CD45RA⁺CD26⁺CD39⁻,
the population of Foxp3⁺ T cells having the following phenotype: CD45RA⁺CD26⁻CD39⁻,
the population of Foxp3⁺ T cells having the following phenotype: CD45RA⁺CD26⁺CD39⁺,
the population of Foxp3⁺ T cells having the following phenotype: CD45RA⁺CD26⁻CD39⁺, and
the population of Foxp3⁺ T cells having the following phenotype: CD45RA⁻CD26⁺CD39⁻,
the population of Foxp3⁺ T cells having the following phenotype: CD45RA⁻CD26⁻CD39⁻,
the population of Foxp3+ T cells (M3) having the following phenotype: CD45RA⁻CD26⁺CD39⁺, and
the population of Foxp3⁺ T cells having the following phenotype: CD45RA⁻CD26⁻CD39⁺.

In some embodiments, said population of Foxp3⁺ T cells are CD4⁺ or CD8⁺.

In some embodiments, said populations of Foxp3⁺ T cells may be CD25⁺ or CD25⁻.

In some embodiments, said populations of Foxp3⁺ T cells are CD127^{-/low}.

Accordingly, the combination of phenotypic markers such as CD3, CD4, CD8, CD45RA, CD25, C26, CD39 and CD127 are particularly suitable for characterising populations of Foxp3+ T. More particularly, the combination of said phenotypic markers are particularly suitable for characterising populations of nTreg cells.

In particular, the inventors characterized the following population of CD25⁺⁻ nTregs cells:
the population of CD4⁺ Foxp3⁺ regulatory T cells (N1) having the following phenotype: CD25⁺CD45RA⁺CD26⁺CD39⁻,
the population of CD4⁺ Foxp3⁺ regulatory T cells (N2) having the following phenotype: CD25⁺CD45RA⁺CD26⁻CD39⁻,
the population of CD4⁺ Foxp3⁺ regulatory T cells (N3) having the following phenotype: CD25⁺CD45RA⁺CD26⁺CD39⁺,
the population of CD4⁺ Foxp3⁺ regulatory T cells (N4) having the following phenotype: CD25⁺CD45RA⁺CD26⁻CD39⁺⁻,
the population of CD4⁺ Foxp3⁺ regulatory T cells (M1) having the following phenotype: CD25⁺CD45RA⁻CD26⁺CD39⁻,
the population of CD4⁺ Foxp3⁺ regulatory T cells (M2) having the following phenotype: CD25⁺CD45RA⁻CD26⁻CD39⁻,
the population of CD4⁺ Foxp3⁺ regulatory T cells (M3) having the following phenotype: CD25⁺CD45RA⁻CD26⁺CD39⁺, and
the population of CD4⁺ Foxp3⁺ regulatory T cells (M4) having the following phenotype: CD25⁺CD45RA⁻CD26⁻CD39⁺.

In some embodiments, said populations of CD25⁺ (M1, M2, M3, M4, N1, N2, N3, and N4) nTregs cells are CD127^{-/low}.

Furthermore, the inventors further characterized the existence of populations of nTregs variant that abnormally loses the expression of CD25. In particular, the inventors characterized the following population of CD25⁻ nTregs cells:
the population of CD4⁺ Foxp3⁺ regulatory T cells (N1′) having the following phenotype: CD25⁻CD45RA⁺CD26⁺CD39⁻.
the population of CD4⁺ Foxp3⁺ regulatory T cells (N2′) having the following phenotype: CD25⁻CD45RA⁺CD26⁻CD39⁻.
the population of CD4⁺ Foxp3⁺ regulatory T cells (N3′) having the following phenotype: CD25−CD45RA⁺CD26⁺CD39⁺.
the population of CD4⁺ Foxp3⁺ regulatory T cells (N4′) having the following phenotype: CD25⁻CD45RA⁺CD26⁻CD39⁺.
the population of CD4⁺ Foxp3⁺ regulatory T cells (M1′) having the following phenotype: CD25⁻CD45RA⁻CD26⁺CD39⁻.
the population of CD4⁺ Foxp3⁺ regulatory T cells (M2′) having the following phenotype: CD25⁻CD45RA⁻CD26⁻CD39⁻.
the population of CD4⁺ Foxp3⁺ regulatory T cells (M3′) having the following phenotype: CD25⁻CD45RA⁻CD26⁺CD39⁺.
the population of CD4⁺ Foxp3⁺ regulatory T cells (M4′) having the following phenotype: CD25⁻CD45RA⁻CD26⁻CD39⁺.

In some embodiments, said populations of CD25⁻ (M1′, M2′, M3′, M4′, N1′, N2′, N′3, and N4′) nTregs cells variant are CD127^{-/low}.

The present invention thus relates to an isolated population of nTregs cells as described above.

In some embodiments, the present invention also relates to an isolated population of CD4⁺Foxp3⁺ cells.

In some embodiments, said isolated population of CD4⁺Foxp3⁺ cells are CD3⁺.

In some embodiments, said isolated population of CD4⁺Foxp3⁺ cells are CD127^{-/low}.

In other embodiments, said isolated population of CD4⁺Foxp3⁺ cells are CD127⁺.

In some embodiments, said isolated population of CD4⁺Foxp3⁺ cells are CD25⁺.

In some embodiments, said isolated population of CD4⁺Foxp3⁺ cells are CD25⁻.

The present invention also relates to a method of determining the presence of a population of Foxp3+ T cells as described above comprising i) detecting the expression of the phenotypic markers CD45RA, CD25, CD26 and CD39 in said population of Foxp3+ T cells and ii) determining to which category belongs said population of Foxp3+ T cells.

More particularly, the present invention also relates to a method of assessing the maturation stage of a population of Foxp3+ T cells comprising i) detecting the expression of the phenotypic markers CD45RA, CD25, CD26 and CD39 in said population of Foxp3+ T cells and ii) determining to which category of maturation stage, (e.g. N1, N2, N3, N4, M1, M2, M3 and M4) belongs said population of Foxp3+ T cells.

Methods for determining the expression level are well-known from the skilled artisan. In some embodiments, the expression of the phenotypic marker is assessed at the mRNA level. Methods for assessing the transcription level of a molecule are well known in the prior art. Examples of such methods include, but are not limited to, RT-PCR, RT-qPCR, Northern Blot, hybridization techniques such as, for example, use of microarrays, and combination thereof including but not limited to, hybridization of amplicons obtained by RT-PCR, sequencing such as, for example, next-generation DNA sequencing (NGS) or RNA-seq (also known as “Whole Transcriptome Shotgun Sequencing”) and the like. In some embodiments, the expression of the phenotypic marker is assessed at the protein level. Methods for determining a protein level in a sample are well-known in the art. Examples of such methods include, but are not limited to, immunohistochemistry, Multiplex methods (Luminex), western blot, enzyme-linked immunosorbent assay (ELISA), sandwich ELISA, fluorescent-linked immunosorbent assay (FLISA), enzyme immunoassay (EIA), radioimmunoassay (MA), flow cytometry (FACS) and the like.

In some embodiments, determining the expression level of at least one phenotypic marker corresponds to detecting and/or quantifying binding of a ligand to said phenotypic marker. In some embodiments, said ligand is an antibody specific of said phenotypic marker, and the method of the invention comprises detecting and/or quantifying a complex formed between said antibody and said phenotypic marker. In some embodiments, determining the expression level of phenotypic markers is conducted by flow cytometry, immunofluorescence or image analysis, for example high content analysis. Preferably, the determination of the expression level of phenotypic markers is conducted by flow cytometry. In some embodiments, before conducting flow cytometry analysis, cells are fixed and permeabilized, thereby allowing detecting intracellular proteins (e.g. Foxp3). The expression level of the phenotypic marker of interest is typically determined by comparing the Median Fluorescence Intensity (MFI) of the cells from the cell population stained with fluorescently labeled antibody specific for this marker to the fluorescence intensity (FI) of the cells from the same cell population stained with fluorescently labeled antibody with an irrelevant specificity but with the same isotype, the same fluorescent probe and originated from the same specie (referred as Isotype control). The cells from the population stained with fluorescently labeled antibody specific for this marker and that show equivalent MFI or a lower MFI than the cells stained with the isotype controls are not expressing this marker and then are designated (−) or negative. The cells from the population stained with fluorescently labeled antibody specific for this marker and that show a MFI value superior to the cells stained with the isotype controls are expressing this marker and then are designated (+) or positive. In some embodiments, determining the expression level of a phenotypic marker in a cell population comprises determining the percentage of cells of the cell population expressing the phenotypic marker (i.e. cells “+” for the phenotypic marker). Preferably, said percentage of cells expressing the phenotypic marker is measured by fluorescence activated cell sorting (FACS). In some embodiments, the expression level of cell maker of interest is “low” by comparison with the expression level of that cell marker in the population of cells being analyzed as a whole. More particularly, the term “lo” refers to a distinct population of cells being analyzed as a whole. Accordingly, FACS can be used with the methods described herein to isolate and detect the population of cells of the present invention. FACS typically involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Laluza . . . ), data input devices, e.g., interface ports, a mouse, a keyboard, etc., fluid handling components, power sources, etc. Typically, the population of cells is contacted with a panel of antibodies specific for the specific phenotypic markers of interest (i.e. CD25, CD45RA, CD26 and CD36). Typically, the antibodies are labelled with a tag to facilitate the isolation and detection of population of cells of the interest. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650. The aforementioned assays may involve the binding of the antibodies to a solid support. The solid surface could be a microtitration plate coated with the antibodies.

Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In some embodiments, fluorescent beads are those contained in TruCount™ tubes, available from Becton Dickinson Biosciences, (San Jose, Calif.). Intracellular flow cytometry typically involves the permeabilization and fixation of the cells. Any convenient means of permeabilizing and fixing the cells may be used in practicing the methods. For example permeabilizing agent typically include saponin, methanol, Tween® 20, Triton X-100™.

In some embodiments, the isolated populations of the invention has been frozen and tawed.

In some embodiments, the expression of a least one additional phenotypic marker is determined. In some embodiments, the marker is selected from the group consisting of Foxp3, CD3, CD8, CD4, CDS, CD2, CD103, CD119, CD120a, CD120b, CD122, CD127, CD134, CD14, CD152, CD154, CD178, CD183, CD184, CD19, CD1a, CD210, CD27, CD28, CD3, CD32, CD4, CD44, CD45RO, CD47, CD49d, CD54, CD56, CD62L, CD69, CD7, CD8, CD80, CD83, CD86, CD95, CD97, CD98, CXCR6, GITR, HLA-DR, IFNalphaRII, IL-18Rbeta, KIR-NKAT2, TGFRII, GZMB, GLNY, TBX21, IRF1, IFNG, CXCL9, CXCL10, CXCR3, CXCR6, IL-18, IL-18Rbeta, Fractalkine, IL-23, IL-31, IL-15, IL-7, MIG, Perforin, TCRalpha/beta, TCRgamma/delta, LAT, ZAP70, CCR5, and CR7. In some embodiments, the additional phenotypic marker is selected from the group consisting of ACE, ACTB, AGTR1 , AGTR2, APC, APOA1, ARF1, AXIN1, BAX, BCL2, BCL2L1, CXCR5, BMP2, BRCA1, BTLA, C3, CASP3, CASP9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCLS, CCL7, CCL8, CCNB1, CCND1, CCNE1, CCR1, CCR10, CCR2, CCR3, CCR4, CCR5, CCR6, CCR7, CCR8, CCR9, CCRL2, CD154 , CD19, CD1a, CD2, CD226, CD244, PDCD1LG1, CD28, CD34, CD36, CD38, CD3E, CD3G, CD3Z, CD4, CD40LG, CDS, CD54, CD6, CD68, CD69, CLIP, CD80, CD83, SLAMF5, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNNB1, CT SC, CX3CL1, CX3CR1, CXCL1, CXCL10, CXCL11, CXCL12, CXCL13, CXCL14, CXCL16, CXCL2, CXCL3, CXCL5, CXCL6, CXCL9, CXCR3, CXCR4, CXCR6, CYP1A2, CYP7A1, DCC, DCN, DEFA6, DICER1, DKK1, Dok-1, Dok-2, DOK6, DVL1, E2F4, EBI3, ECE1, ECGF1, EDN1, EGF, EGFR, EIF4E , CD105, ENPEP, ERBB2, EREG, FCGR3A, CGR3B, FN1, FOXP3, FYN, FZD1, GAPD, GLI2, GNLY, GOLPH4, GRB2, GSK3B, GSTP1, GUSB, GZMA, GZMB, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, Dl, ifna1, ifnal7, ifna2, ifna5, ifna6, ifna8, IFNAR1, IFNAR2, IFNG, IFNGR1, IFNGR2, IGF1, IHH, IKBKB, IL10, IL12A, IL12B, IL12RB1, IL12RB2, IL13, IL13RA2, IL15, IL15RA, IL17, IL17R, IL17RB, IL18, ILIA, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin, alpha E (antigen CD103, human mucosal lymphocyte, antigen 1; alpha polypeptide),Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7, MAF, MAP2K1, MDM2 , MICA, MICB, MKI67, MMP12, MMP9, MTA1, MTSS1, MYC , MYD88, MYH6, NCAM1, NFATC1, NKG7, NLK, NOS2A, P2X7, PDCD1, PECAM-, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO , RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STAT6, STK36, TAP1, TAP2, TBX21, TCF7, TERT, TFRC, TGFA, TGFB1, TGFBR1, TGFBR2, TIMP3, TLR1, TLR10, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2. In some embodiments, the additional phenotypic marker is an immune checkpoint protein selected from the group consisting of CD40 (CD40 molecule, TNF receptor superfamily member 5), CD274 (CD274 molecule, also known as B7-H; B7H1; PDL1; PD-L1; PDCD1L1; PDCD1LG1), ICOS (inducible T-cell co-stimulator), TNFRSF9 (tumor necrosis factor receptor superfamily member 9, also known as ILA; 4-1BB; CD137; CDw137), TNFRSF18 (tumor necrosis factor receptor superfamily member 18, also known as AITR; GITR; CD357; GITR-D), LAG3(lymphocyte-activation gene 3), HAVCR2 (hepatitis A virus cellular receptor 2), TNFRSF4 (tumor necrosis factor receptor superfamily member 4), CD276(CD276 molecule), CTLA-4 (cytotoxic T-lymphocyte-associated protein 4), PDCD1LG2 (programmed cell death 1 ligand 2, also known as B7DC; Btdc; PDL2; CD273; PD-L2; PDCD1L2; bA574F11.2), VTCN1 (V-set domain containing T cell activation inhibitor 1, also known as B7H4), PDCD1 (programmed cell death 1, also known as PD1; PD-1; CD279; SLEB2; hPD-1; hPD-1; hSLE1), BTLA (B and T lymphocyte associated), CD28 (CD28 molecule), TIGIT (T cell immunoreceptor with Ig and ITIM domains), C10orf54 (chromosome 10 open reading frame 54) and CD27 (CD27 molecule).

The methods as above described are also suitable for detecting and quantifying the population of Foxp3+ T cells of the present invention (e.g. N1, N2, N3, N4, M1, M2, M3, and M4) in a sample.

In some embodiments, said sample is a body fluid sample, as described hereinabove. In some embodiments, said sample is a blood sample, more preferably a PBMC sample. In some embodiments, said sample is a tissue sample, as described hereinabove.

Diagnostic Methods of the Present Invention:

As demonstrated in the EXAMPLE, quantifying the populations of Foxp3+ T cells according to the present invention, in particular populations N1, N2, N3, N4, M1, M2, M3, and M4, is particularly suitable for diagnostic purposes.

In some embodiments, said particular populations N1, N2, N3, N4, M1, M2, M3, and M4 cells are quantified among CD25⁺ nTreg cells. In other embodiments, said particular populations N1′, N2′, N3′, N4′, M1′, M2′, M3′, and M4′ cells are quantified among CD25⁻nTreg cells. In other embodiment, said particular populations are quantified among CD25⁺ nTreg cells and CD25⁻ nTreg cells.

Methods for determining whether a subject suffers from an impaired immune response and/or immunosenescence.

In particular, quantifying said populations may be suitable for determining whether a subject suffers from an impaired immune response and/or immunosenescence.

Accordingly the present invention relates to a method of determining whether a subject suffers from an impaired immune response and/or immunosenescence comprising i) quantifying the amount of at least one populations of Foxp3+ T cells of the present invention in a sample obtained from the subject, ii) comparing the amount quantified at step i) with a predetermined reference value wherein detecting differential between said amount and said predetermined reference value indicates whether the subject suffers from an impaired immune response and/or immunosenescence.

In some embodiments, at least 2, 3, 4, 5, 6, 7, 8, 9, 10, 11, 12, 13, 14, 15; or 16 populations of Foxp3+ T cells are quantified.

In some embodiments, the quantification is absolute or relative. In some embodiments, the quantification is relative to another population of cells that can be another population of Foxp3+ T of the present invention. Thus in some embodiments, a ratio between at least 2 populations of cells is calculated and compared to a predetermined reference value wherein detecting differential between said amount and said predetermined reference value indicates whether the subject suffers from an impaired immune response and/or immunosenescence.

Methods for Determining Whether a Subject Has or is at Risk of Having an Autoimmune Inflammatory Disease, an Infectious Disease or a Cancer:

In particular, quantifying said populations may be suitable for determining whether a subject has or is at risk of having an autoimmune inflammatory disease, an infectious disease or a cancer.

Accordingly the present invention relates to a method of determining whether a subject has or is at risk of having an autoimmune inflammatory disease, an infectious disease or a cancer comprising i) quantifying the amount of at least one populations of Foxp3+ T cells of the present invention in a sample obtained from the subject, ii) comparing the amount quantified at step i) with a predetermined reference value wherein detecting differential between said amount and said predetermined reference value indicates whether the subject has or is at risk of having an autoimmune inflammatory disease, an infectious disease or a cancer.

In some embodiments, at least 2, 3, 4, 5, 6, 7, 8, 9, 10, 11, 12, 13, 14, 15; or 16 populations of Foxp3+ T cells are quantified.

In some embodiments, the quantification is absolute or relative. In some embodiments, the quantification is relative to another population of cells that can be another population of Foxp3+ T of the present invention. Thus in some embodiments, a ratio between at least 2 populations of cells is calculated and compared to a predetermined reference value wherein detecting differential between said amount and said predetermined reference value indicates whether the subject has or is at risk of having an autoimmune inflammatory disease, an infectious disease or a cancer.

Methods for Determining Whether a Patient Suffering from an Autoimmune Disease, an Infectious Disease or a Cancer Achieves a Therapeutic Response with a Treatment:

Quantifying the population of Foxp3+ T of the present invention is also particularly suitable for determining whether a patient suffering from an autoimmune disease or a cancer achieves a therapeutic response with a treatment. Quantifying the population of Foxp3+ T of the present invention is thus particularly suitable for discriminating responder from non-responder.

Accordingly the present invention relates to a method of determining whether a patient suffering from an autoimmune inflammatory disease, an infectious disease or a cancer achieves a therapeutic response with a treatment comprising i) quantifying the amount of at least one populations of Foxp3+ T cells of the present invention in a sample obtained from the subject and ii) and comparing the amount quantified at step i) with a predetermined reference value wherein detecting differential between said amount and said predetermined reference value indicates whether the patient achieves or does not achieve a therapeutic response with the treatment.

In some embodiments, at least 2, 3, 4, 5, 6, 7, 8, 9, 10, 11, 12, 13, 14, 15; or 16 populations of Foxp3+ T cells are quantified.

In some embodiments, the quantification is absolute or relative. In some embodiments, the quantification is relative to another population of cells that can be another population of Foxp3+ T of the present invention. Thus in some embodiments, a ratio between at least 2 populations of cells is calculated and compared to a predetermined reference value wherein detecting differential between said amount and said predetermined reference value indicates whether the subject achieves or does not achieve a therapeutic response.

Methods for Determining Whether a Patient Suffering from an Autoimmune Inflammatory Disease, an Infectious Disease or a Cancer is at Risk of Relapse:

The present invention also relates to a method of determining whether a patient suffering from an autoimmune inflammatory disease, an infectious disease or a cancer is at risk of relapse comprising i) quantifying the amount of at least one population of Foxp3+ T cells of the present invention in a sample obtained from the subject, and ii) comparing the amount quantified at step i) with a predetermined reference value wherein detecting differential between said amount and said predetermined reference value indicates whether the patient is or is not at risk of relapse.

In some embodiments, at least 2, 3, 4, 5, 6, 7, 8, 9, 10, 11, 12, 13, 14, 15; or 16 populations of Foxp3+ T cells are quantified.

In some embodiments, the quantification is absolute or relative. In some embodiments, the quantification is relative to another population of cells that can be another population of Foxp3+ T of the present invention. Thus in some embodiments, a ratio between at least 2 populations of cells is calculated and compared to a predetermined reference value wherein said amount and said predetermined reference value indicates whether the patient is or is not at risk of relapse.

According to the present invention, the treatment consists in any method or drug that could be suitable for the treatment of the autoimmune inflammatory disease, the infectious disease or the cancer.

Methods for Predicting the Survival Time of Patients Suffering from Cancer:

A further object of the present invention relates to a method for predicting the survival time of a subject suffering from a cancer comprising i) quantifying the amount of at least one population of Foxp3+ T cells of the present invention in a sample obtained from the subject, and ii) comparing the amount quantified at step i) with a predetermined reference value wherein detecting differential between said amount and said predetermined reference value indicates whether the patient will have a short or long survival time.

The method of the present invention is particularly suitable for predicting the duration of the overall survival (OS), progression-free survival (PFS) and/or the disease-free survival (DFS) of the cancer patient. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the patient will have a survival time that will be lower than the median (or mean) observed in the general population of patients suffering from said cancer. When the patient will have a short survival time, it is meant that the patient will have a “poor prognosis”. Inversely, the expression “long survival time” indicates that the patient will have a survival time that will be higher than the median (or mean) observed in the general population of patients suffering from said cancer. When the patient will have a long survival time, it is meant that the patient will have a “good prognosis”.

In some embodiments, at least 2, 3, 4, 5, 6, 7, 8, 9, 10, 11, 12, 13, 14, 15; or 16 populations of Foxp3+ T cells are quantified.

In some embodiments, the quantification is absolute or relative. In some embodiments, the quantification is relative to another population of cells that can be another population of Foxp3+ T of the present invention. Thus in some embodiments, a ratio between at least 2 populations of cells is calculated and compared to a predetermined reference value wherein said amount and said predetermined reference value indicates whether the patient is or is not at risk of relapse.

Treatments of Autoimmune Inflammatory Disease:

In some embodiments, the patient suffering from an autoimmune inflammatory disease is treated with an immunosuppressive drug. Immunosuppressive drugs include, without limitation thiopurine drugs such as azathioprine (AZA) and metabolites thereof; nucleoside triphosphate inhibitors such as mycophenolic acid (Cellcept) and its derivative (Myfortic); derivatives thereof; prodrugs thereof; and combinations thereof. Other examples include but are not limited to 6-mercaptopurine (“6-MP”), cyclophosphamide, mycophenolate, prednisolone, sirolimus, dexamethasone, rapamycin, FK506, mizoribine, azothioprine and tacrolimus.

In some embodiments the immunosuppressive drug is a calcineurin inhibitor. As used herein, the term “calcineurin inhibitor” has its general meaning in the art and refers to substances which block calcineurin (i.e. calcium/calmodulin-regulated protein phosphatase involved in intracellular signalling) dephosphorylation of appropriate substrates, by targeting calcineurin phosphatase (PP2B, PP3), a cellular enzyme that is involved in gene regulation. A calcineurin inhibitor of the present invention is typically an immunophilin-binding compound having calcineurin inhibitory activity. Immunophilin-binding calcineurin inhibitors are compounds forming calcineurin inhibiting complexes with immunophilins, e.g. cyclophilin and macrophilin. Examples of cyclophilin-binding calcineurin inhibitors are cyclosporines or cyclosporine derivatives (hereinafter cyclosporines) and examples of macrophilin-binding calcineurin inhibitors are ascomycin (FR 520) and ascomycin derivatives (hereinafter ascomycins). A wide range of ascomycin derivatives are known, which either are naturally occurring among fungal species or are obtainable by manipulation of fermentation procedures or by chemical derivatization. Ascomycin-type macrolides include ascomycin, tacrolimus (FK506), sirolimus and pimecrolimus. Cyclosporine, originally extracted from the soil fungus Potypaciadium infilatum, has a cyclic 11-amino acid structure and includes e.g. Cyclosporines A through I, such as Cyclosporine A, B, C, D and G. Voclosporin is a next-generation calcineurin inhibitor that is a more potent and less toxic semi-synthetic derivative of cyclosporine A. In some embodiments, the calcineurin inhibitor of the present invention is the trans-version of voclosporin, trans-ISA247 (Cas number 368455-04-3) which is described in, for example, US Patent Publication No.: 2006/0217309, which is hereby incorporated herein by reference. Further compositions of voclosporin are described, for example, in U.S. Pat. No. 7,060,672, which is hereby incorporated herein by reference. Tacrolimus (FK506) is another calcineurin inhibitor which is also a fungal product, but has a macrolide lactone structure. Sirolimus (rapamycin) is a microbial product isolated from the actinomycete Streptomyces hygroscopicus. Sirolimus binds to an immunophilin (FK-binding protein 12, FKBP12) forming a complex, which inhibits the mammalian target of rapamycin (mTOR) pathway through directly binding the mTOR Complex1 (mTORC1). Pimecrolimus is also a calcineurin inhibitor. Calcineurin inhibitors such as cyclosporine A, voclosporin, ascomycin, tacrolimus, pimecrolimus, an analog thereof, or a pharmaceutically acceptable salt thereof, can be utilized in a mixed micellar composition of the present disclosure.

In some embodiments, the immunosuppressive drug is a corticosteroid. As used, the term “corticosteroids” has its general meaning in the art and refers to class of active ingredients having a hydrogenated cyclopentoperhydrophenanthrene ring system endowed with an anti-inflammatory activity. Corticosteroid drugs typically include cortisone, cortisol, hydrocortisone (11β, 17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16α-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

In some embodiments, the immunosuppressive drug is a B cell depleting agent. As used herein, the term “B cell depleting agent” refers to any agent that is capable of triggering lymphodepletion of B cells. In some embodiments, the B cell depleting agent is an antibody having specificity for CD20. Examples of antibodies having specificity for CD20 include: “C2B8” which is now called “Rituximab” (“RITUXAN®”) (U.S. Pat. No. 5,736,137, expressly incorporated herein by reference), a chimaeric pan-B antibody targeting CD20; the yttrium-[90]-labeled 2B8 murine antibody designated “Y2B8” or “Ibritumomab Tiuxetan” ZEVALIN® (U.S. Pat. No. 5,736,137, expressly incorporated herein by reference), a murine IgG1 kappa mAb covalently linked to MX-DTPA for chelating to yttrium-[90]; murine IgG2a “BI,” also called “Tositumomab,” optionally labeled with radioactive 1311 to generate the “1311-B1” antibody (iodine 131 tositumomab, BEXXAR™) (U.S. Pat. No. 5,595,721, expressly incorporated herein by reference); murine monoclonal antibody “1F5” (Press et al. Blood 69 (2):584-591 (1987) and variants thereof including “framework patched” or humanized 1F5 (WO03/002607, Leung, S.; ATCC deposit HB-96450); murine 2H7 and chimeric 2H7 antibody (U.S. Pat. No. 5,677,180, expressly incorporated herein by reference); humanized 2H7, also known as ocrelizumab (PRO-70769); Ofatumumab (Arzerra), a fully human IgG1 against a novel epitope on CD20 huMax-CD20 (Genmab, Denmark; WO2004/035607 (U.S. Ser. No. 10/687,799, expressly incorporated herein by reference)); AME-133 (ocaratuzumab; Applied Molecular Evolution), a a fully-humanized and optimized IgG1 mAb against CD20; A20 antibody or variants thereof such as chimeric or humanized A20 antibody (cA20, hA20, respectively) (U.S. Ser. No. 10/366,709, expressly incorporated herein by reference, Immunomedics); and monoclonal antibodies L27, G28-2, 93-1B3, B-CI or NU-B2 available from the International Leukocyte Typing Workshop (Valentine et al, In: Leukocyte Typing III (McMichael, Ed., p. 440, Oxford University Press (1987)). Further, suitable antibodies include e.g. antibody GA101 (obinutuzumab), a third generation humanized anti-CD20-antibody of Biogen Idec/Genentech/Roche. Moreover, BLX-301 of Biolex Therapeutics, a humanized anti CD20 with optimized glycosylation or Veltuzumab (hA20), a 2nd-generation humanized antibody specific for CD20 of Immunomedics or DXL625, derivatives of veltuzumab, such as the bispecific hexavalent antibodies of IBC Pharmaceuticals (Immunomedics) which are comprised of a divalent anti-CD20 IgG of veltuzumab and a pair of stabilized dimers of Fab derived from milatuzumab, an anti-CD20 mAb enhanced with InNexus' Dynamic Cross Linking technology, of Inexus Biotechnology both are humanized anti-CD20 antibodies are suitable. Further suitable antibodies are BM-ca (a humanized antibody specific for CD20 (Int J. Oncol. 2011 February; 38(2):335-44)), C2H7 (a chimeric antibody specific for CD20 (Mol Immunol. 2008 May; 45(10):2861-8)), PRO131921 (a third generation antibody specific for CD20 developed by Genentech), Reditux (a biosimilar version of rituximab developed by Dr Reddy's), PBO-326 (a biosimilar version of rituximab developed by Probiomed), a biosimilar version of rituximab developed by Zenotech, TL-011 (a biosimilar version of rituximab developed by Teva), CMAB304 (a biosimilar version of rituximab developed by Shanghai CP Guojian), GP-2013 (a biosimilar version of rituximab developed by Sandoz (Novartis)), SAIT-101 (a biosimilar version of rituximab developed by Samsung BioLogics), a biosimilar version of rituximab developed by Intas Biopharmaceuticals, CT-P10), a biosimilar version of rituximab developed by Celltrion), a biosimilar version of rituximab developed by Biocad, Ublituximab (LFB-R603, a transgenically produced mAb targeting CD20 developed by GTC Biotherapeutics (LFB Biotechnologies)), PF-05280586 (presumed to be a biosimilar version of rituximab developed by Pfizer), Lymphomun (Bi-20, a trifunctional anti-CD20 and anti-CD3 antibody, developed by Trion Pharma), a biosimilar version of rituximab developed by Natco

Pharma, a biosimilar version of rituximab developed by iBio, a bio similar version of rituximab developed by Gedeon Richter/Stada, a biosimilar version of rituximab developed by Curaxys, a biosimilar version of rituximab developed by Coherus Biosciences/Daiichi Sankyo, a biosimilar version of rituximab developed by BioXpress, BT-D004 (a biosimilar version of rituximab developed by Protheon), AP-052 (a biosimilar version of rituximab developed by Aprogen), a biosimilar version of ofatumumab developed by BioXpress, MG-1106 (a biosimilar version of rituximab developed by Green Cross), IBI-301 (a humanized monoclonal antibody against CD20 developed by Innovent Biologics), BVX-20 (a humanized mAb against the CD20 developed by Vaccinex), 20-C2-2b (a bispecific mAb-IFNalpha that targets CD20 and human leukocyte antigen-DR (HLA-DR) developed by Immunomedics), MEDI-552 (developed by MedImmune/AstraZeneca), the anti-CD20/streptavidin conjugates developed by NeoRx (now Poniard Pharmaceuticals), the 2nd generation anti-CD20 human antibodies developed by Favrille (now MMRGlobal), TRU-015, an antibody specific for CD20 fragment developed by Trubion/Emergent BioSolutions, as well as other precloinical approaches by various companies and entities. All aforementioned publications, references, patents and patent applications are incorporated by reference in their entireties. All antibodies disclosed in therein may be used within the present invention. In some embodiments, the method of the present invention is particularly suitable for determining whether a patient suffering from an inflammatory autoimmune disease achieves a therapeutic response with a treatment. In particular the monitoring method of the present invention comprises providing a sample of the patient after a period of treatment and concluding that the patient achieves a therapeutic response when the immunoreceptor of the leukocytes present in the sample returns to an inhibiting steady state of ITAM signaling or concluding that the patient does not achieve a response when the immunoreceptor of the leukocytes present in the sample are maintained in their activating steady state of ITAM signaling.

In some embodiments, the patient suffering from an autoimmune inflammatory disease is treated with a biotherapy for inhibiting the activity of an inflammatory cytokine such as TNF-alpha, IL-1beta, IL-6, IL-8, IL-17 . . . Typically the biotherapy consists in administering to the patient a therapeutically effective amount of an antibody or decoy receptor protein having specificity for the inflammatory cytokine or the receptor of the inflammatory cytokine. For example, the drug is an anti-TNFalpha drug. As used herein, the term “anti-TNFα drug” is intended to encompass agents including proteins, antibodies, antibody fragments, fusion proteins (e.g., Ig fusion proteins or Fc fusion proteins), multivalent binding proteins (e.g., DVD Ig), small molecule TNFα antagonists and similar naturally- or normaturally-occurring molecules, and/or recombinant and/or engineered forms thereof, that, directly or indirectly, inhibit TNFα activity, such as by inhibiting interaction of TNFα with a cell surface receptor for TNFα, inhibiting TNFα protein production, inhibiting TNFα gene expression, inhibiting TNFα secretion from cells, inhibiting TNFα receptor signaling or any other means resulting in decreased TNFα activity in a subject. The term “anti-TNFα drug” preferably includes agents which interfere with TNFα activity. Examples of anti-TNFα drugs include, without limitation, infliximab (REMICADE™, Johnson and Johnson), human anti-TNF monoclonal antibody adalimumab (D2E7/HUMIRA™, Abbott Laboratories), etanercept (ENBREL™, Amgen), certolizumab pegol (CIMZIA®, UCB, Inc.), golimumab (SIMPONI®; CNTO 148), CDP 571 (Celltech), CDP 870 (Celltech), as well as other compounds which inhibit TNFα activity, such that when administered to a subject in which TNFα activity is detrimental, the disorder (i.e. acute severe colitis) could be treated.

Treatments of Infectious Diseases:

In some embodiments, the patients suffering from a bacterial infection is administered with an antibiotic.

In some embodiments, the antibiotic is selected from the group consisting of aminoglycosides, beta lactams, quinolones or fluoroquinolones, macrolides, sulfonamides, sulfamethaxozoles, tetracyclines, streptogramins, oxazolidinones (such as linezolid), rifamycins, glycopeptides, polymixins, lipo-peptide antibiotics. Aminoglycoside antibiotics for use in the invention include amikacin (Amikin®), gentamicin (Garamycin®), kanamycin (Kantrex®), neomycin (Mycifradin®), netilmicin (Netromycin®), paromomycin (Humatin®), streptomycin, and tobramycin (TOBI Solution®, TobraDex®). Macrolides for use in the invention include azithromycin (Zithromax®), clarithromycin (Biaxin®), dirithromycin (Dynabac®), erythromycin, clindamycin, josamycin, roxithromycin and lincomycin. Representative ketolides for use in the invention include telithromycin (formerly known as HMR-3647), HMR 3004, HMR 3647, cethromycin, EDP-420, and ABT-773. Quinolones for use in the invention span first, second, third and fourth generation quinolones, including fluoroquinolones. Such compounds include nalidixic acid, cinoxacin, oxolinic acid, flumequine, pipemidic acid, rosoxacin, norfloxacin, lomefloxacin, ofloxacin, enrofloxacin, ciprofloxacin, enoxacin, amifloxacin, fleroxacin, gatifloxacin, gemifloxacin, clinafloxacin, sitafloxacin, pefloxacin, rufloxacin, sparfloxacin, temafloxacin, tosufloxacin, grepafloxacin, levofloxacin, moxifloxacin, and trovafloxacin. Sulfonamide antibiotics for use in the invention include the following: mafenide, phtalylsulfathiazole, succinylsulfathiazole, sulfacetamide, sulfadiazine, sulfadoxine, sulfamazone, sulfamethazine, sulfamethoxazole, sulfametopirazine, sulfametoxypiridazine, sulfametrol, sulfamonomethoxine, sulfamylon, sulfanilamide, sulfaquinoxaline, sulfasalazine, sulfathiazole, sulfisoxazole, sulfisoxazole diolamine, and sulfaguanidine. Beta-lactams include 2-(3-alanyl)clavam, 2-hydroxymethylclavam, 7-methoxycephalosporin, epi-thienamycin, acetyl-thienamycin, amoxicillin, apalcillin, aspoxicillin, azidocillin, azlocillin, aztreonam, bacampicillin, blapenem, carbenicillin, carfecillin, carindacillin, carpetimycin A and B, cefacetril, cefaclor, cefadroxil, cefalexin, cefaloglycin, cefaloridine, cefalotin, cefamandole, cefapirin, cefatrizine, cefazedone, cefazolin, cefbuperazone, cefcapene, cefdinir, cefditoren, cefepime, cefetamet, cefixime, cefinenoxime, cefinetazole, cefminox, cefmolexin, cefodizime, cefonicid, cefoperazone, ceforamide, cefoselis, cefotaxime, cefotetan, cefotiam, cefoxitin, cefozopran, cefpiramide, cefpirome, cefpodoxime, cefprozil, cefquinome, cefradine, cefroxadine, cefsulodin, ceftazidime, cefteram, ceftezole, ceftibuten, ceftizoxime, ceftriaxone, cefuroxime, cephalosporin C, cephamycin A, cephamycin C, cephalothin, chitinovorin A, chitinovorin B, chitinovorin C, ciclacillin, clometocillin, cloxacillin, cycloserine, deoxy pluracidomycin B and C, dicloxacillin, dihydro pluracidomycin C, epicillin, epithienamycin D, E, and F, ertapenem, faropenem, flomoxef, flucloxacillin, hetacillin, imipenem, lenampicillin, loracarbef, mecillinam, meropenem, metampicillin, meticillin (also referred to as methicillin), mezlocillin, moxalactam, nafcillin, northienamycin, oxacillin, panipenem, penamecillin, penicillin G, N, and V, phenethicillin, piperacillin, povampicillin, pivcefalexin, povmecillinam, pivmecillinam, pluracidomycin B, C, and D, propicillin, sarmoxicillin, sulbactam, sultamicillin, talampicillin, temocillin, terconazole, thienamycin, andticarcillin. In certain embodiments, the peptide antibiotic is a lipopeptide antibiotic such as colistin, daptomycin, surfactin, friulimicin, aculeacin A, iturin A, and tsushimycin.

In some embodiments, the patient suffering from a viral infection is administered with an anti-viral compound.

Conventional antiviral treatments include, but are not limited to (1) Amantadine and rimantadine, which combat influenza and act on penetration/uncoating; (2) Pleconaril, which works against rhinoviruses, which cause the common cold; (3) nucleotide or nucleoside analogues, such as acyclovir, zidovudine (AZT), lamivudine; (4) drugs based on “antisense” molecules, such as fomivirsen; (5) ribozyme antivirals; (6) protease inhibitors; (7) assembly inhibitors, such as Rifampicin; (8) release phase inhibitors, such as zanamivir (Relenza) and oseltamivir (Tamiflu); (9) drugs which stimulate the immune system, such as interferons, which inhibit viral synthesis in infected cells (e.g., interferon alpha), and synthetic antibodies (A monoclonal drug is now being sold to help fight respiratory syncytial virus in babies, and antibodies purified from infected individuals are also used as a treatment for hepatitis B). Examples of antiviral drugs include, but are not limited to, Abacavir, Aciclovir, Acyclovir, Adefovir, Amantadine, Amprenavir, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Entry inhibitors, Famciclovir, Fixed dose combination (antiretroviral), Fomivirsen, Fosamprenavir, Foscarnet, Fosfonet, Fusion inhibitor, Ganciclovir, Ibacitabine, Immunovir, Idoxuridine, Imiquimod, Indinavir, Inosine, Integrase inhibitor, Interferon type III, Interferon type II, Interferon type I, Interferon, Lamivudine, Lopinavir, Loviride, Maraviroc, Molixan (NOV-205), Moroxydine, Nelfinavir, Nevirapine, Nexavir, Nucleoside analogues, Oseltamivir (Tamiflu®), Peginterferon alfa-2a, Penciclovir, Peramivir, Pleconaril, Podophyllotoxin, Protease inhibitor (pharmacology), Raltegravir, Reverse transcriptase inhibitor, Ribavirin, Rimantadine, Ritonavir, Saquinavir, Stavudine, Synergistic enhancer (antiretroviral), Tenofovir, Tenofovir disoproxil, Tipranavir, Trifluridine, Trizivir, Tromantadine, Truvada, Valaciclovir (Valtrex®), Valganciclovir, Vicriviroc, Vidarabine, Viramidine, Zalcitabine, Zanamivir (Relenza®), and Zidovudine.

In some embodiments, the patient suffering from a fungal infection is administered with an anti-fungal infection.

Suitable antifungal agents are selected from the group of Miconazole, etoconazole, Clotrimazole, Econazole, Bifonazole, Butoconazole, Fenticonazole, Isoconazole, Oxiconazole, Sertaconazole, Sulconazole, Tioconazole, Griseofulvin, Fluconazole Fosfluconazole, Itraconazole, Posaconazole, Voriconazole, Amorolfine, Butenafine, Naftifine, Terbinafine, Terbinafine, Natamycin, Nystatin, Amphotericin B, Thiabendazole, Anidulafungin, Caspofungin, Micafungin and Flucytosin.

In some embodiments, the patient suffering from a cancer is administered with radiotherapy, chemotherapy and/or immunotherapy.

In some embodiments, the patient is administered with a chemotherapeutic agent selected from the group consisting of alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g. , calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP- 16); ifosfamide; mitoxantrone; vincristine; vinorelbine; novantrone; teniposide; edatrexate; daunomycin; aminopterin; xeloda; ibandronate; irinotecan (e.g., CPT-1 1); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the immunotherapy consists in administering the patient with at least one immune checkpoint inhibitor. As used herein, the term “immune checkpoint inhibitor” has its general meaning in the art and refers to any compound inhibiting the function of an immune inhibitory checkpoint protein. As used herein the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al. , 2011. Nature 480:480- 489). Examples of inhibitory checkpoint molecules include A2AR, B7-H3, B7-H4, BTLA, CTLA-4, CD277, IDO, KIR, PD-1, LAG-3, TIM-3 and VISTA. Inhibition includes reduction of function and full blockade. Preferred immune checkpoint inhibitors are antibodies that specifically recognize immune checkpoint proteins. A number of immune checkpoint inhibitors are known and in analogy of these known immune checkpoint protein inhibitors, alternative immune checkpoint inhibitors may be developed in the (near) future. The immune checkpoint inhibitors include peptides, antibodies, nucleic acid molecules and small molecules. Examples of immune checkpoint inhibitor includes PD-1 antagonist, PD-L1 antagonist, PD-L2 antagonist CTLA-4 antagonist, VISTA antagonist, TIM-3 antagonist, LAG-3 antagonist, IDO antagonist, KIR2D antagonist, A2AR antagonist, B7-H3 antagonist, B7-H4 antagonist, and BTLA antagonist.

In some embodiments, PD-1 (Programmed Death-1) axis antagonists include PD-1 antagonist (for example anti-PD-1 antibody), PD-L1 (Programmed Death Ligand-1) antagonist (for example anti-PD-L1 antibody) and PD-L2 (Programmed Death Ligand-2) antagonist (for example anti-PD-L2 antibody). In some embodiments, the anti-PD-1 antibody is selected from the group consisting of MDX-1106 (also known as Nivolumab, MDX-1106-04, ONO-4538, BMS-936558, and Opdivo®), Merck 3475 (also known as Pembrolizumab, MK-3475, Lambrolizumab, Keytruda®, and SCH-900475), and CT-011 (also known as Pidilizumab, hBAT, and hBAT-1). In some embodiments, the PD-1 binding antagonist is AMP-224 (also known as B7-DCIg). In some embodiments, the anti-PD-L1 antibody is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55. S70 is an anti-PD-L1 described in WO 2010/077634 A1. MEDI4736 is an anti-PD-L1 antibody described in WO2011/066389 and US2013/034559. MDX-1106, also known as MDX-1106-04, ONO-4538 or BMS-936558, is an anti-PD-1 antibody described in U.S. Pat. No. 8,008,449 and WO2006/121168. Merck 3745, also known as MK-3475 or SCH-900475, is an anti-PD-1 antibody described in U.S. Pat. No. 8,345,509 and WO2009/114335. CT-011 (Pidizilumab), also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342. Atezolimumab is an anti-PD-L1 antibody described in U.S. Pat. No. 8,217,149. Avelumab is an anti-PD-L1 antibody described in US 20140341917. CA-170 is a PD-1 antagonist described in WO2015033301 & WO2015033299. Other anti-PD-1 antibodies are disclosed in U.S. Pat. No. 8,609,089, US 2010028330, and/or US 20120114649. In some embodiments, the PD-1 inhibitor is an anti-PD-1 antibody chosen from Nivolumab, Pembrolizumab or Pidilizumab. In some embodiments, PD-L1 antagonist is selected from the group comprising of Avelumab, BMS-936559, CA-170, Durvalumab, MCLA-145, SP142, STI-A1011, STIA1012, STI-A1010, STI-A1014, A110, KY1003 and Atezolimumab and the preferred one is Avelumab, Durvalumab or Atezolimumab.

In some embodiments, CTLA-4 (Cytotoxic T-Lymphocyte Antigen-4) antagonists are selected from the group consisting of anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (Ipilimumab), Tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B. Additional CTLA-4 antibodies are described in U.S. Pat. Nos. 5,811,097; 5,855,887; 6,051,227; and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17): 10067-10071 (1998); Camacho et al., J. Clin: Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281. A preferred clinical CTLA-4 antibody is human monoclonal antibody (also referred to as MDX-010 and Ipilimumab with CAS No. 477202-00-9 and available from Medarex, Inc., Bloomsbury, N.J.) is disclosed in WO 01/14424. With regard to CTLA-4 antagonist (antibodies), these are known and include Tremelimumab (CP-675,206) and Ipilimumab.

In some embodiments, the immunotherapy consists in administering to the patient a combination of a CTLA-4 antagonist and a PD-1 antagonist.

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM-3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94). As used herein, the term “TIM-3” has its general meaning in the art and refers to T cell immunoglobulin and mucin domain-containing molecule 3. The natural ligand of TIM-3 is galectin 9 (Ga19). Accordingly, the term “TIM-3 inhibitor” as used herein refers to a compound, substance or composition that can inhibit the function of TIM-3. For example, the inhibitor can inhibit the expression or activity of TIM-3, modulate or block the TIM-3 signaling pathway and/or block the binding of TIM-3 to galectin-9. Antibodies having specificity for TIM-3 are well known in the art and typically those described in WO2011155607, WO2013006490 and WO2010117057.

In some embodiments, the immune checkpoint inhibitor is an IDO inhibitor. Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6- fluoro-tryptophan, 4-methyl-tryptophan, 5-methyl tryptophan, 6-methyl-tryptophan, 5-methoxy-tryptophan, 5 -hydroxy-tryptophan, indole 3-carbinol, 3,3′- diindolylmethane, epigallocatechin gallate, 5-Br-4-Cl-indoxyl 1,3-diacetate, 9- vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. Preferably the IDO inhibitor is selected from 1-methyl-tryptophan, β-(3- benzofuranyl)-alanine, 6-nitro-L-tryptophan, 3-Amino-naphtoic acid and β-[3-benzo(b)thienyl] -alanine or a derivative or prodrug thereof.

In some embodiments, immunotherapy consists in administering the patient with a therapeutically effective amount of at least one cytokine. A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors. Interferons (IFNs) contemplated by the present invention include the common types of IFNs, IFN-alpha (IFN-a), IFN-beta (IFN-beta) and IFN-gamma (IFN-y). IFNs boost the immune system and/or stimulating natural killer (NK) cells, T cells and macrophages. Recombinant IFN-alpha is available commercially as Roferon (Roche Pharmaceuticals) and Intron A (Schering Corporation). Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention. Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin). Non-cytokine adjuvants suitable for use in the combinations of the present invention include, but are not limited to, Levamisole, alum hydroxide (alum), Calmette-Guerin bacillus (ACG), incomplete Freund's Adjuvant (IFA), QS-21, DETOX, Keyhole limpet hemocyanin (KLH) and dinitrophenyl (DNP).

In some embodiments, immunotherapy involves the use of cancer vaccines. Cancer vaccines have been developed that comprise whole cancer cells, parts of cancer cells or one or more antigens derived from cancer cells. Cancer vaccines, alone or in combination with one or more immuno- or chemotherapeutic agents are being investigated in the treatment of several types of cancer including melanoma, renal cancer, ovarian cancer, breast cancer, colorectal cancer, and lung cancer. Non-specific immunotherapeutics are useful in combination with cancer vaccines in order to enhance the body's immune response.

Predetermined Reference Values:

Typically, the predetermined reference value is a threshold value or a cut-off value that can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER. SAS, CREATE-ROC. SAS, GB STAT VI0.0 (Dynamic Microsystems, Inc. Silver Spring, Md., USA), etc.

In some embodiments, the predetermined reference value is the amount of the population in healthy individuals.

In some embodiments, the ratio between the population CD127⁺ and the population CD127⁻ is determined among CD3⁺CD4⁻Foxp3⁺ cells, wherein higher is the ratio CD127⁻/CD127⁻, higher is the probability that the subject has or is at risk of having an autoimmune inflammatory disease.

In some embodiments, the ratio between the population CD25⁺ and the population CD25⁻ is determined among CD3⁺CD4⁻Foxp3⁺ cells, wherein lower is the ratio CD25⁻/CD25⁻, higher is the probability that the subject has or is at risk of having an autoimmune inflammatory disease.

In some embodiments, an accumulation of Foxp3⁻CD25⁻ and Foxp3⁻/CD127⁺ Foxp3+ T cells is associated with autoimmune inflammatory diseases.

In some embodiments, an accumulation of the (M4) population indicates that the subject has or is at risk of having an autoimmune inflammatory disease. In some embodiments, the ratio between the population (M4) and (M1) is determined, wherein higher is the ratio M4/M1, higher is the probability that the subject has or is at risk of having an autoimmune inflammatory disease.

In some embodiments, an accumulation of the N2, N3 and N4 naive populations and/or a decrease of the N1 naive population indicates that the subject has or is at risk of a cancer. In some embodiments, the ratio between the population (N4) and (N1) is determined, wherein higher is the ratio N4/N1, higher is the probability that the subject has or is at risk of having a cancer.

In some embodiments, the ratio between the population (N4) and (N1) is determined, wherein lower is the ratio N4/N1, higher is the probability that the subject has a long survival time.

In some embodiments, the ratio between the population (M4) and (M1) is determined, wherein lower is the ratio M4/M1, higher is the probability that the subject has a long survival time.

Methods for Determining Whether a Patient Suffering from Cancer is Eligible to a Treatment with a CD39 Inhibitor:

In particular, quantifying said populations, especially CD39+ cells may be suitable for determining whether a patient suffering from cancer is eligible to a treatment with a CD39 inhibitor.

The present invention thus relates to a method of determining whether a patient suffering from cancer is eligible to a treatment with a CD39 inhibitor i) comprising quantifying the amount of a population of CD39+ cell of the present invention and ii) comparing the amount quantified at step i) with a predetermined reference value wherein detecting differential between said amount and said predetermined reference value indicates whether the subject is or is not eligible to a treatment with a CD39 inhibitor.

In some embodiments, the quantification is absolute or relative. In some embodiments, the quantification is relative to another population of cells that can be another population of Foxp3+ T of the present invention. Thus in some embodiments, a ratio between at least 2 populations of cells is calculated and compared to a predetermined reference value wherein detecting differential between said amount and said predetermined reference value indicates whether the patient is or is not eligible to a treatment with a CD39 inhibitor.

In some embodiment, the CD39 inhibitor is an anti-CD39 antibody. Monoclonal antibodies that are CD39 inhibitors are well known in the art and includes those described in the international patent application WO 2009095478, WO2012085132 and in the following publications: Bonnefoy N, Bastid J, Alberici G, Bensussan A, Eliaou J F. CD39: A complementary target to immune checkpoints to counteract tumor-mediated immunosuppression. Oncoimmunology. 2015 Feb. 3;4(5):e1003015. eCollection 2015 May; Bastid J, Regairaz A, Bonnefoy N, Dejou C, Giustiniani J, Laheurte C, Cochaud S, Laprevotte E, Funck-Brentano E, Hemon P, Gros L, Bee N, Larroque C, Alberici G, Bensussan A, Eliaou J F. Inhibition of CD39 enzymatic function at the surface of tumor cells alleviates their immunosuppressive activity. Cancer Immunol Res. 2015 Mar;3(3):254-65.

Kits of the Present Invention:

A further object of the invention relates to kit comprising means for performing the methods of the present invention. Typically, the kit comprises means for detection of the presence or absence of the phenotypic markers of interest. In some embodiments, said means are antibodies as described above. In some embodiments, the kit comprises a panel of antibodies specific for CD25, CD45RA, CD26 and CD39. In some embodiments, the kit further comprises an antibody specific for Foxp3 and/or an antibody specific for CD4. In some embodiments, the kit further comprises antibodies specific for CD3, CD4, Foxp3. In some embodiments, the kit further comprises antibodies specific for CD3, CD4, Foxp3, CD127. In some embodiments, the kit further comprises at least one antibody specific for a phenotypic marker selected from the group consisting of CD3, CD8, CD4, CD5, CD2, CD103, CD119, CD120a, CD120b, CD122, CD127, CD134, CD14, CD152, CD154, CD178, CD183, CD184, CD19, CD1a, CD210, CD27, CD28, CD3, CD32, CD4, CD44, CD45Ro, CD47, CD49d, CD54, CD56, CD62L, CD69, CD7, CD8, CD80, CD83, CD86, CD95, CD97, CD98, CXCR6, GITR, HLA-DR, IFNalphaRII, IL-18Rbeta, KIR-NKAT2, TGFRII, GZMB, GLNY, TBX21, IRF1, IFNG, CXCL9, CXCL10, CXCR3, CXCR6, IL-18, IL-18Rbeta, Fractalkine, IL-23, IL-31, IL-15, IL-7, MIG, Perforin, TCRalpha/beta, TCRgamma/delta, LAT, ZAP70, CCR5, and CR7. In some embodiments, the additional phenotypic marker is selected from the group consisting of ACE, ACTB, AGTR1, AGTR2, APC, APOA1, ARF1, AXIN1, BAX, BCL2, BCL2L1, CXCR5, BMP2, BRCA1, BTLA, C3, CASP3, CASP9, CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCLS, CCL7, CCL8, CCNB1, CCND1, CCNE1, CCR1, CCR10, CCR2, CCR3, CCR4, CCR5, CCR6, CCR7, CCR8, CCR9, CCRL2, CD154 , CD19, CD1a, CD2, CD226, CD244, PDCD1LG1, CD28, CD34, CD36, CD38, CD3E, CD3G, CD3Z, CD4, CD4OLG, CD5, CD54, CD6, CD68, CD69, CLIP, CD80, CD83, SLAMFS, CD86, CD8A, CDH1, CDH7, CDK2, CDK4, CDKN1A, CDKN1B, CDKN2A, CDKN2B, CEACAM1, COL4A5, CREBBP, CRLF2, CSF1, CSF2, CSF3, CTLA4, CTNNB1, CT SC, CX3CL1, CX3CR1, CXCL1, CXCL10, CXCL11, CXCL12, CXCL13, CXCL14, CXCL16, CXCL2, CXCL3, CXCLS, CXCL6, CXCL9, CXCR3, CXCR4, CXCR6, CYP1A2, CYP7A1, DCC, DCN, DEFA6, DICER1, DKK1, Dok-1, Dok-2, DOK6, DVL1, E2F4, EBI3, ECE1, ECGF1, EDN1, EGF, EGFR, EIF4E , CD105, ENPEP, ERBB2, EREG, FCGR3A, CGR3B, FN1, FOXP3, FYN, FZD1, GAPD, GLI2, GNLY, GOLPH4, GRB2, GSK3B, GSTP1, GUSB, GZMA, GZMB, GZMH, GZMK, HLA-B, HLA-C, HLA-, MA, HLA-DMB, HLA-DOA, HLA-DOB, HLA-DPA1, HLA-DQA2, HLA-DRA, HLX1, HMOX1, HRAS, HSPB3, HUWE1, ICAM1, ICAM-2, ICOS, ID1, ifna1, ifnal7, ifna2, ifna5, ifna6, ifna8, IFNAR1, IFNAR2, IFNG, IFNGR1, IFNGR2, IGF1, IHH, IKBKB, IL10, IL12A, IL12B, IL12RB1, IL12RB2, IL13, IL13RA2, IL15, IL15RA, IL17, IL17R, IL17RB, IL18, ILIA, IL1B, IL1R1, IL2, IL21, IL21R, IL23A, IL23R, IL24, IL27, IL2RA, IL2RB, IL2RG, IL3, IL31RA, IL4, IL4RA, IL5, IL6, IL7, IL7RA, IL8, CXCR1, CXCR2, IL9, IL9R, IRF1, ISGF3G, ITGA4, ITGA7, integrin, alpha E (antigen CD103, human mucosal lymphocyte , antigen 1; alpha polypeptide),Gene hCG33203, ITGB3, JAK2, JAK3, KLRB1, KLRC4, KLRF1, KLRG1, KRAS, LAG3, LAIR2, LEF1, LGALS9, LILRB3, LRP2, LTA, SLAMF3, MADCAM1, MADH3, MADH7,MAF, MAP2K1, MDM2 , MICA, MICB, MKI67, MMP12, MMP9, MTA1, MTSS1, MYC , MYD88, MYH6, NCAM1, NFATC1, NKG7, NLK, NOS2A, P2X7, PDCD1, PECAM-, CXCL4, PGK1, PIAS1, PIAS2, PIAS3, PIAS4, PLAT, PML, PP1A, CXCL7, PPP2CA, PRF1, PROM1, PSMB5, PTCH, PTGS2, PTP4A3, PTPN6, PTPRC, RAB23, RAC/RHO , RAC2, RAF, RB1, RBL1, REN, Drosha, SELE, SELL, SELP, SERPINE1, SFRP1, SIRP beta 1, SKI, SLAMF1, SLAMF6, SLAMF7, SLAMF8, SMAD2, SMAD4, SMO, SMOH, SMURF1, SOCS1, SOCS2, SOCS3, SOCS4, SOCS5, SOCS6, SOCS7, SOD1, SOD2, SOD3, SOS1, SOX17, CD43, ST14, STAM, STAT1, STAT2, STAT3, STAT4, STAT5A, STAT5B, STATE, STK36, TAP1, TAP2, TBX21, TCF7, TERT, TFRC, TGFA, TGFB1, TGFBR1, TGFBR2, TIMP3, TLR1, TLR10, TLR2, TLR3, TLR4, TLR5, TLR6, TLR7, TLR8, TLR9, TNF, TNFRSF10A, TNFRSF11A, TNFRSF18, TNFRSF1A, TNFRSF1B, OX-40, TNFRSF5, TNFRSF6, TNFRSF7, TNFRSF8, TNFRSF9, TNFSF10, TNFSF6, TOB1, TP53, TSLP, VCAM1, VEGF, WIF1, WNT1, WNT4, XCL1, XCR1, ZAP70 and ZIC2. In some embodiments, the kit of the present invention further comprises at least one antibody specific for a phenotypic marker selected from the group consisting of CD40, CD274, TNFRSF9, TNFRSF18, LAG3, HAVCR2, TNFRSF4, CD276, CTLA-4, PDCD1LG2, VTCN1, PDCD1, BTLA, CD28, TIGIT, C10orf54 and CD27. In some embodiments, these antibodies are labelled as described above. Typically, the kits described above will also comprise one or more other containers, containing for example, wash reagents, and/or other reagents capable of quantitatively detecting the presence of bound antibodies. The kit also contains agents suitable for performing intracellular flow cytometry such as agents for permeabilization and fixation of cells. Typically, compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the sample, a container which contains the antibody(s) used in the assay, containers which contain wash reagents (such as phosphate buffered saline, Tris-buffers, and like), and containers which contain the detection reagent.

The invention will be further illustrated by the following figures and examples. However, these examples and figures should not be interpreted in any way as limiting the scope of the present invention.

FIGURES

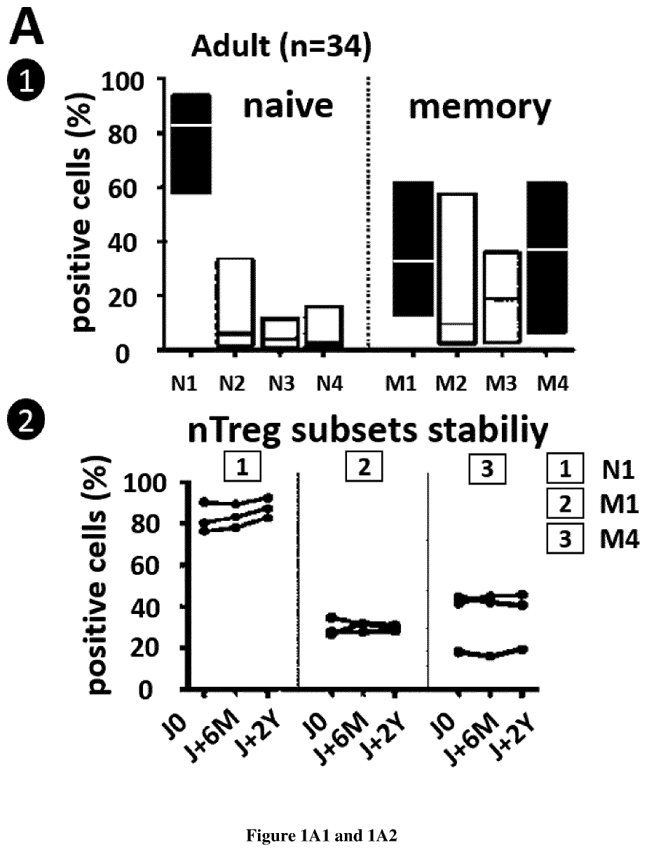

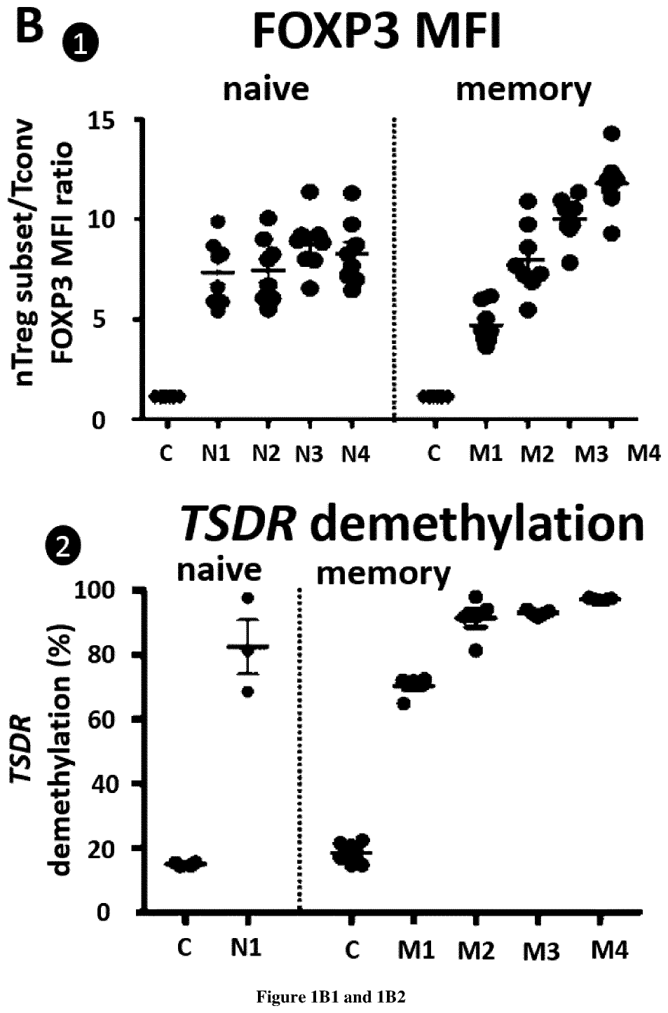

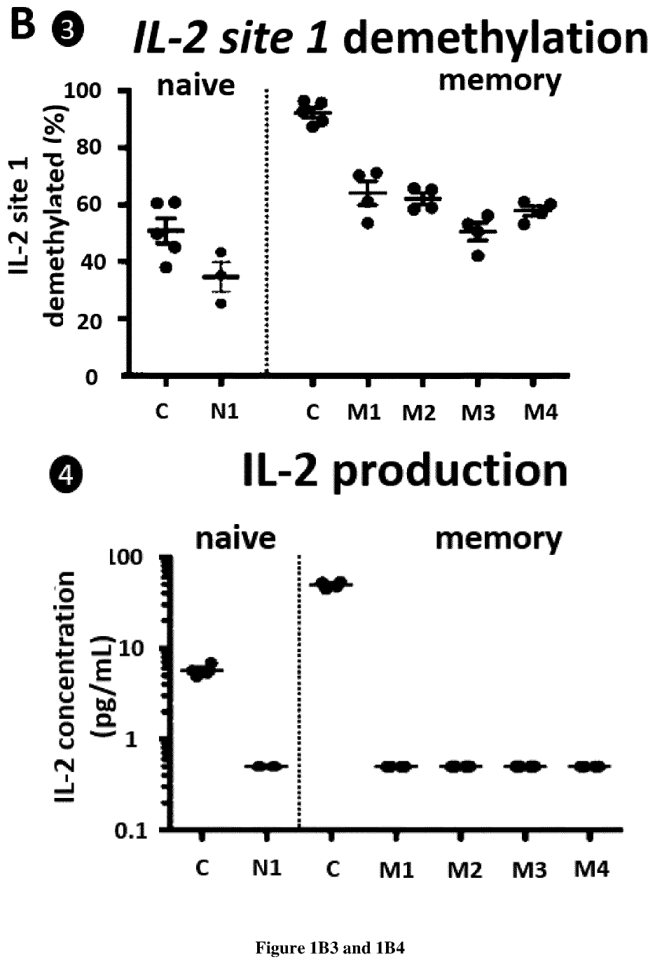

FIG. 1. FOXP3 nTreg heterogeneity in healthy human PBMCs analysis. (A1-A2) Boxplots showing the distribution of the 4 nTreg subsets based on their expression of CD39 and CD26 in naive and memory CD25⁻nTreg variants. Longitudinal analysis of nTreg subsets frequency in 3 individuals for over a 2-year period. (B) Facs sorted nTreg subsets phenotypic, epigenetic and physiologic characteristic s. (B1) Summary plot of MFI ratio of FOXP3 expression on Treg subsets to Tconv cells. Scatterplot indicating FOXP3−TSDR (B2) and CpG site 1 in the IL-2 region promoter (B3) demethylation status of both the 5 major FACS sorted nTreg subsets (N1, M1-4) and the 2 conventional T cells (C, naive and memory) as assessed by bisulfate pyrosequencing. CFSE-labelled nTreg subsets (N1, M1, M4) and Tconv (4×10⁴ per well) were stimulated with 0.5 ug/ml of pbaCD3 in presence of irradiated ΔCD3 feeder. (B4) Measure of IL-2 secretion in culture supernatant from 40h-stimulated T cells by ELISA. (B5) T cell activation status and T cell proliferation were evaluated by the median fluorescence intensity of CD25 expression and the CFSE dilution assay respectively. Data shown are expressed as mean±SEM.

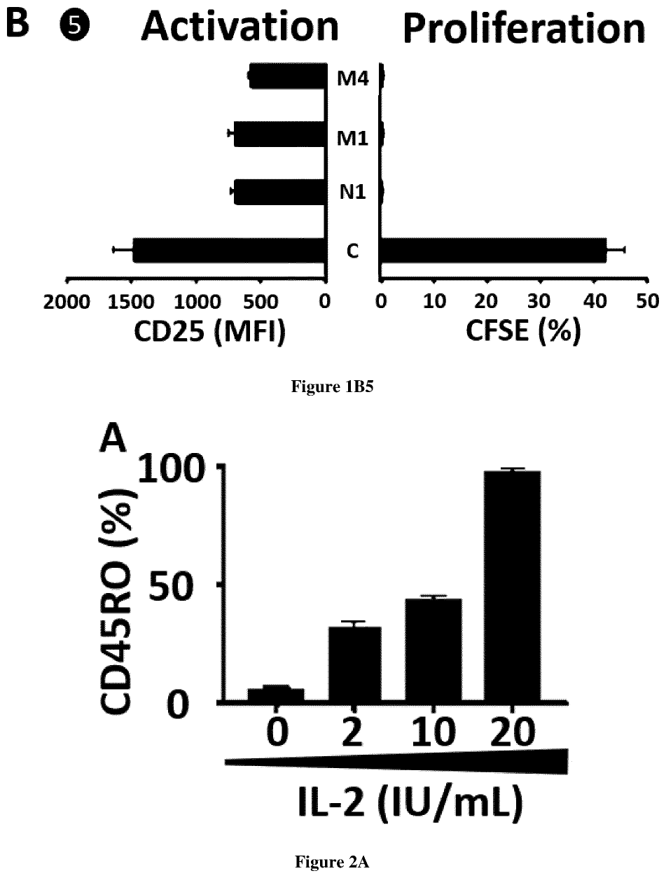

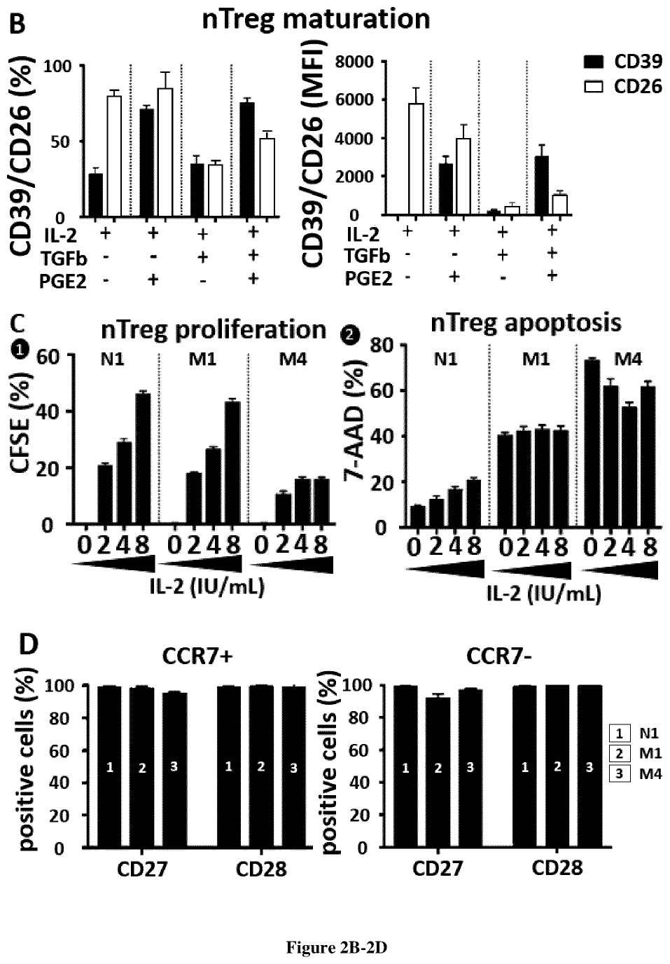

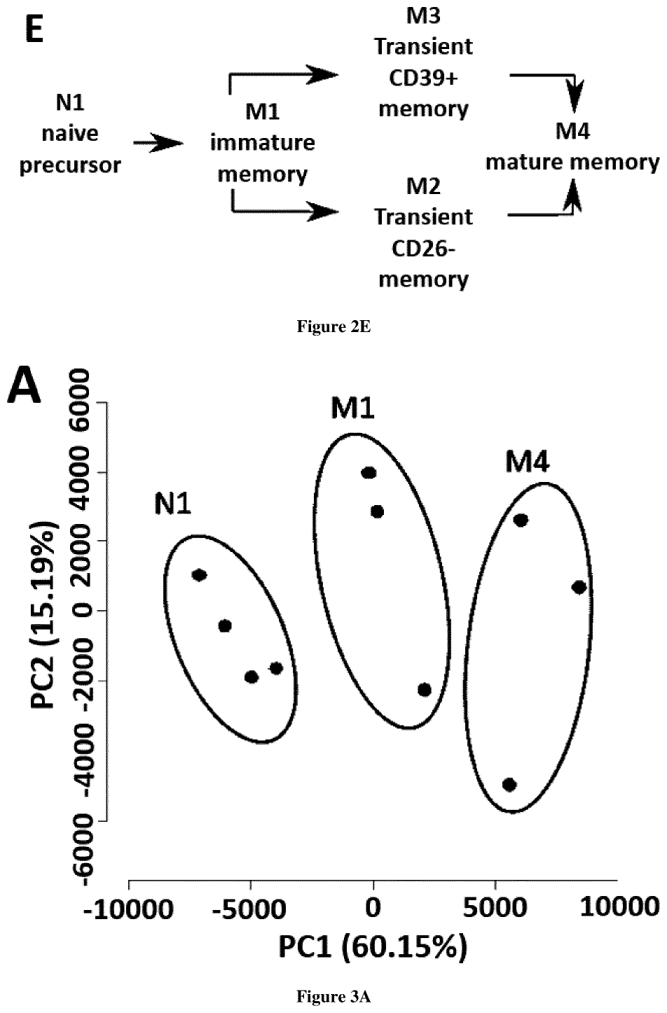

FIG. 2. Microenvironmental context of TCR stimulation governs nTreg subset parental maturation. (A) N1 cells convert into M1 cells after stimulation in vitro. Histogram indicating the percentage of CD45RI expressed by stimulated N1 cells (n=3). (B) M4 cells convert into M1 cells in vitro when stimulated as above in presence of IL-2, TGF-β and PGE2Histograms indicating both the percentage of CD26 and CD39 exhibited by stimulated M1 cells and their MFI (n=3). (C) M4 cells represent a no return differentiation stage. CFSE-labeled nTreg subsets were stimulated as indicated above. (C1) Histogram indicating the percentage of proliferating cells in stimulated cell culture (n=4). (C2) Histogram showing the percentage of 7-AAD positive stimulated nTreg subsets (D) Graphs showing CD27 and CD28 frequency in the 3 nTreg subsets N1, M1 and M4 expressing either CCR7⁺ or CCRT (n=3). (E) Schema of parental maturation process of the nTreg population. Data shown are expressed as mean±SEM.

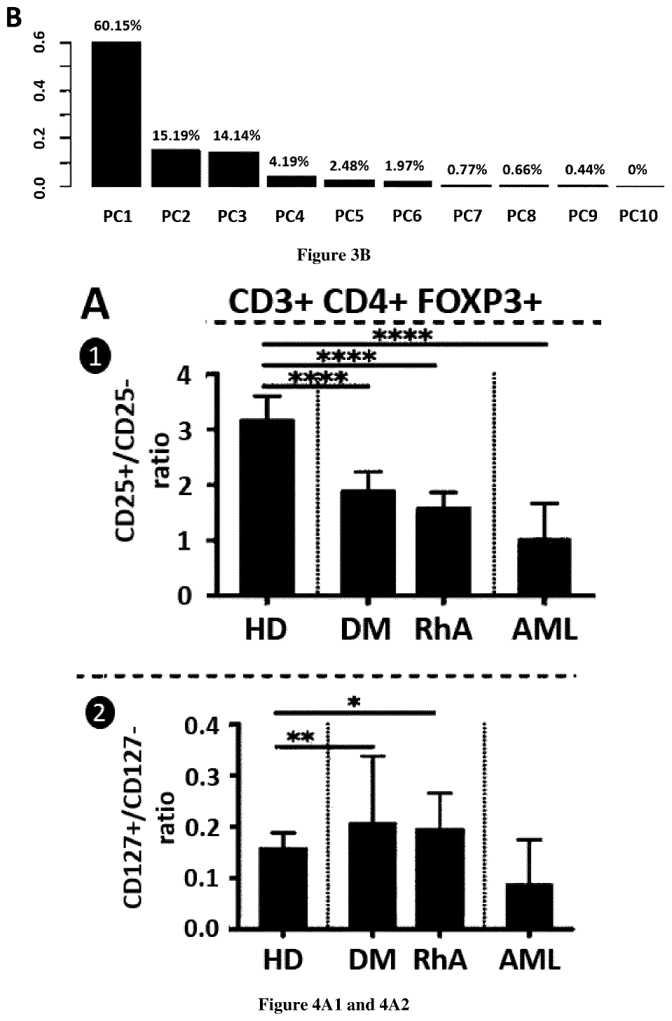

FIG. 3: RNA sequencing analysis confirmed both nTreg subsets heterogeneity and parental maturation. N1, Ml and M4 nTreg populations present distinct transcriptomic profiles. (A-B) Principal component analysis performed on whole transcriptome data of 10 nTregs samples obtained by RNA sequencing experiments including 25313 genes in transcripts per kilobase million (TPM).

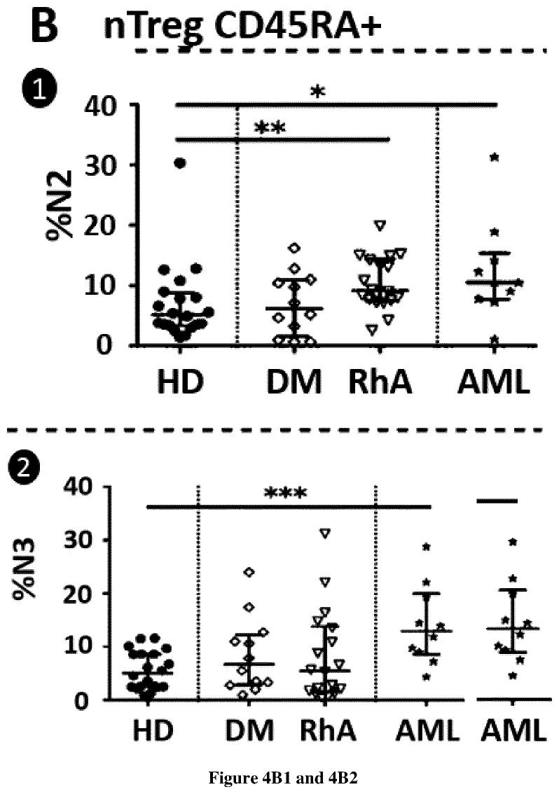

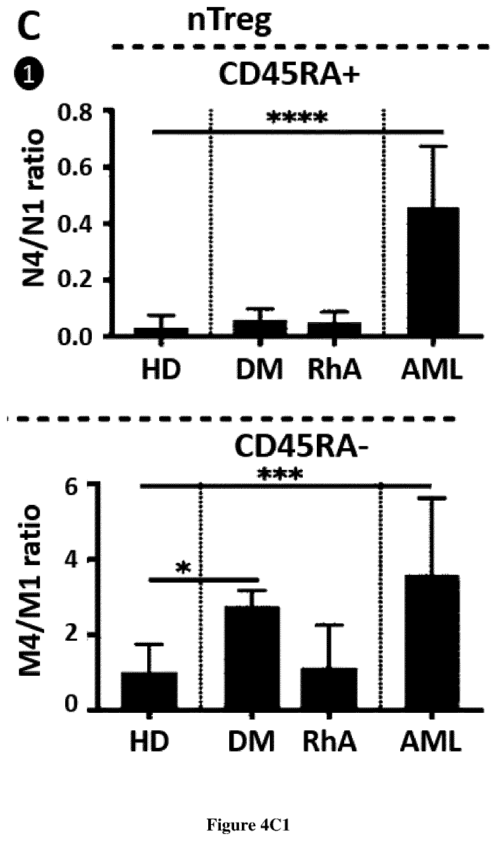

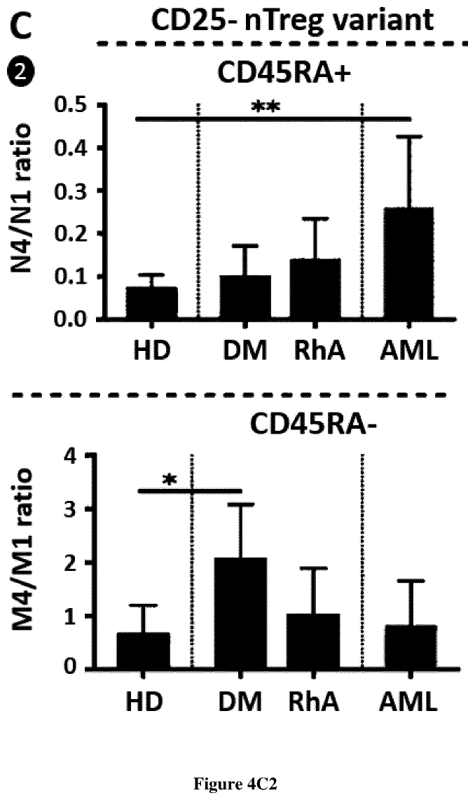

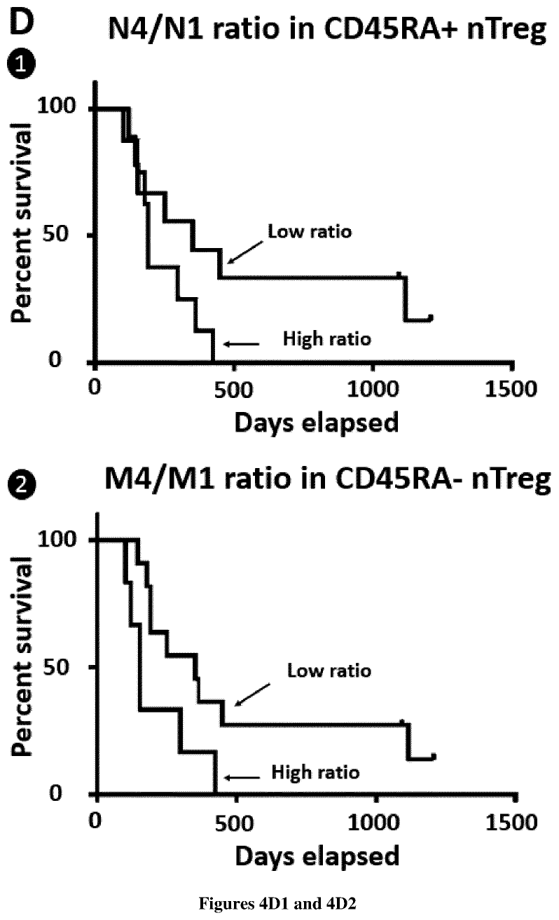

FIG. 4. FOXP3+ regulatory blood sub-population's distribution is modified in auto-immunity and cancer. (A, B, C) Data are presented as median with interquartile range in HD compared to DM, RhA and AML. (A) Histogram of CD25+/CD25− (A1) and CD127⁻/CD127⁻ (A2) frequency ratio among CD4⁻FOXP3⁺ T cells. (B) Scatter plot depict the frequency of N2 population (B1) and N3 population (B2) among nTreg RA⁺. (C) Histograms depict the frequency ratio of N4/N1 and M4/M1 among nTreg CD45RA⁺ and nTreg CD45RA⁻ respectively, defined by FOXP3⁺CD127⁻CD25⁺ (C1). Histograms depict the frequency ratio of N4/N1 and M4/M1 among nTreg variant CD45RA⁺ and nTreg variant CD45RA⁻ respectively, defined by FOXP3⁺CD127⁻CD25− (C2). (D1-2) Kaplan-Meier survival curve representing the survival percentage of relapsing AML patients after HSCT treated by Azacytidine and donor lymphocytes infusion after inclusion. Patients were divided into 2 groups either with low (upper curve) or high (lower curve) ratio. N4/N1 ratio; a 0.2 cut-off was used between high and low ratio (p=0.1) (El). M4/M1 ratio; a 4-cut-off ratio was used between high and low ratio (p=0.01) (E2).

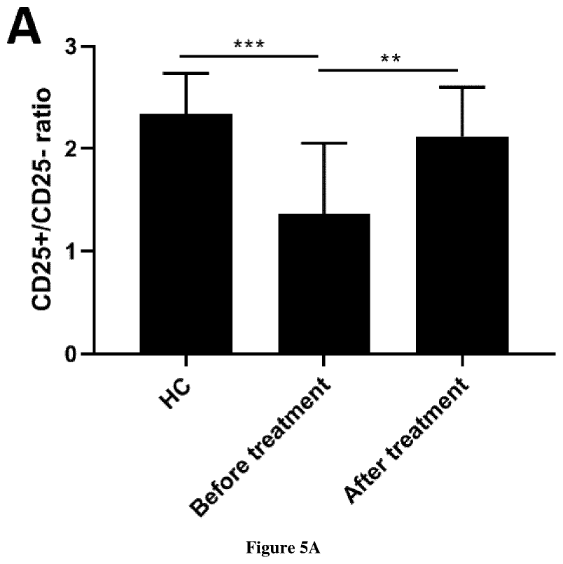

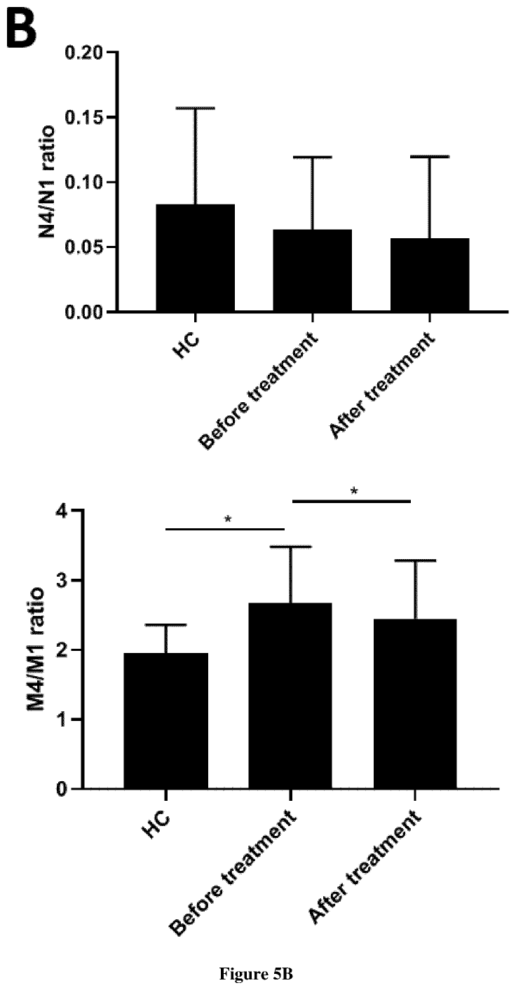

FIG. 5. Evolution of Treg subpopulations after treatment during dermatomyositis. Data are presented as median with interquartile range in HD compared to DM before and after treatment. (A) Histogram of CD25+/CD25− frequency ratio among CD4⁺FOXP3⁺ T cells. (B) Histograms depict the frequency ratio of N4/N1 and M4/M1 among nTreg CD45RA⁺ and nTreg CD45RA⁻ respectively, defined by FOXP3⁺CD127⁻CD25⁺.

### EXAMPLE

I—CD39 CD26 Markers Help Delineate Structural Phenotypic and Genetic Heterogeneity of nTregs in Human Blood.

At a resting stage, nTregs developed in the thymus, are currently characterized by their expression of high levels of CD25, low levels of CD127, expression of the master transcript

FOXP3 (16), a demethylated TSDR (26) and are not able to synthesize IL-2 thereby being functionally anergic (27). In the present study, nTregs identification in healthy human blood was performed using intracellular FOXP3. The cells were then analyzed for the expression of CD25, CD127, CD45RA in combination with the two functional markers CD26 and CD39. The Visne analysis illustrates that CD4⁺ FOXP3⁺ CD127⁻/low exhibit various expression of CD45RA, CD25, CD39 and CD26 markers supporting nTregs heterogeneity (Data not shown). Furthermore, based on CD39 and CD26 expression, the FOXP3⁺ CD127⁻/low CD25+ nTreg population and the FOXP3⁺ CD127^{-/low} CD25⁻nTreg variant population are respectively comprised of 5 major subsets, i.e., naive CD45RA⁺ CD26⁺ CD39⁻(N1), memory CD45RA−CD26⁺ CD39− (M1), CD45RA⁻CD26⁻CD39⁻ (M2), CD45RA⁻CD26⁺ CD39⁺ (M3) and CD45RA⁻CD26⁻CD39⁺ (M4) (FIG. 1A1). All the subsets display high FOXP3 expression (FIG. 1B1), associated with a very high demethylation level of TSDR (FIG. 1B2), and, in contrast to memory Tconv cells, they exhibit a relatively low demethylation level of CpG site 1 in the IL-2 promoter, essential for inducing IL-2 production upon TCR activation (FIG. 1B3) (25). Moreover, nTreg subset cells were functionally in an anergic state, given that, following CD3 stimulation, they were unable to synthesize IL-2 (FIG. 1B4), lost their CD25 activating marker and did not proliferate (FIG. 1B5). These characteristics include these subsets in the nTreg lineage definition. Importantly, whereas nTreg subset distribution is stable in each adult individual for over a two-year period (FIG. 1A2), it varies interindividualy (FIG. 1A1). Also, the nTreg subsets distribution is not gender dependent (Data not shown) but does not vary according to age as illustrated in newborn cord blood (Data not shown) and elderly individuals (Data not shown). The use of intracellular FOXP3⁺ to identify nTregs population in human PBMCs enabled us to highlight other FOXP3⁺ subsets. A subset of CD4⁺ bearing CD8 are present at very low frequencies (1.33% in CD4⁺ FOXP3⁺) (Data not shown). As this discrete population has a similar expression of FOXP3 (Data not shown) and relatively high levels of demethylated TSDR (75%) (Data not shown) as those exhibited by nTregs, they may represent a CD4⁺ CD8⁺ DP nTreg variant. A memory subset of FOXP3⁺ cells with low CD25 expression (19.75%) was also observed (Data not shown). Among this subset, only cells exhibiting CD39⁺ expressed a high demethylation level TSDR associated with a partial demethylation of the CpG site 1 in the IL-2 promoter (Data not shown) and a relatively high expression of FOXP3 (Data not shown). We considered these cells as atypical CD25⁻nTreg variants. Furthermore, the use of FOXP3 as a marker to identify regulatory T cell subtypes enabled us to characterize an activated CD4⁺ FOXP3⁺ CD127⁺ population in resting PBMCs. This cell population represents 7.55% of the FOXP3⁺ CD4⁺ T cells, and were CD39−CD26⁺ (Data not shown) with a low demethylated levels of TSDR (Data not shown) associated with a lower level of FOXP3 expression than that found in nTregs (Data not shown). This was anticipated given that FOXP3 is a regulatory transcript known to be produced in activated memory conventional T cells following their TCR stimulation (17). Interestingly, by using the regulatory marker CD15S (27), we isolated a small subset of cells (13.2%) in this FOXP3+ CD127⁺ population, which express the regulatory protein FOXP3 at a lower MFI level (Data not shown) than that found in nTregs associated with a partially demethylated TSDR (50%) (Data not shown) and other regulatory markers including CD25, CTLA-4, TIGIT, HLA-DR and FCRL3 (Data not shown). It is noteworthy that these cells exhibit, as conventional memory CD4⁺ T cells, a high demethylation level of CpG site 1 in the IL-2 promoter region (Data not shown). These data strongly suggest that the very low percentage of CD127⁺ CD25⁺ FOXP3⁺ present in resting blood cells originate from activated memory conventional CD4⁺ T cells trans-differentiated to expressing regulatory T cells but did not develop from the thymus as the true nTregs are (1).

In summary: 1) based on CD39 / CD26 markers human blood nTreg population can be subdivided into 5 major subsets in which expression of FOXP3 is a necessary but not sufficient characteristic to define nTregs. 2) FOXP3 regulatory transcripts expressed by activated T cells (17) may also be expressed by conventional memory CD4⁺ CD127⁺, though at low levels in healthy human blood.

II—The Microenvironmental Context of TCR Stimulation Governs nTreg Subset Parental Maturation.

nTreg subsets from PBMCs were sorted and cultured separately under various different conditioned media. Naive N1 cells, cultured in the presence of different doses of IL-2, express the CD25 marker, loose their anergic state, and convert to memory cells exhibiting a CD26⁺CD39⁻⁶⁰ M1 profile (FIG. 2A). Memory M1 cells, when TCR stimulated and cultured with IL-2, convert into M4 cells in the presence of TGF-β plus PGE2 (Data not shown). While TGF-β favors the loss of cell surface CD26 marker (72% to 30%), PGE2 enhances the CD39 marker (25% to 67%) (FIG. 2B). The M1 subset cells, when TCR stimulated, in the presence of IL-2, reached a no return stage to CD39⁻ M3 or M4 subsets. FIG. 2C shows that, following TCR stimulation in the presence of IL-2, M4 cells, being at an advanced stage of differentiation, proliferate less (FIG. 2C1) and are more susceptible to apoptosis (FIG. 2C2) than N1 and M1 cells after a 4 day-culture. It is noteworthy that the CCR7 CD27 CD28 based naive/CM/EM/TEMRA cell cycle nomenclature currently acknowledged for CD4⁺ and CD8⁺ T cells (29) does not fit with the nTregs maturation given that CD27 and CD28 markers are maintained all along the nTreg cell cycle (FIG. 2D). FIG. 2.E briefly schematizes the parental maturation process of the nTreg population in healthy individuals: naive precursor (N1) subset cells progress through immature memory (M1) and then to mature memory (M4) via either transient CD26⁻ (M2) or CD39⁺ (M3) subsets.

III—RNA Sequencing Analysis Confirmed Both nTreg Subsets Heterogeneity and Parental Maturation.

A—Heterogeneity of nTreg Populations

In order to characterize the N1, M1 and M4 nTreg populations at a transcriptomic level, RNA sequencing experiments were performed on ten nTregs total RNA samples (4 N1, 3 M1 and 30 3 M4) and generated RNA expression data of 25313 genes in transcripts per kilobase million (TPM). Principal component analysis performed on these data revealed a first component (PC1) explaining 60.15% of the total variance of the transcriptome among the samples which is sufficient to separate them in their three respective groups, i.e. N1, M1 and M4 (FIGS. 3A and 3B). These results were confirmed by unsupervised hierarchical clustering analysis of RNA sequencing data which showed the clustering of the samples in three distinct groups corresponding to N1, M1 and M4, where M1 and M4 samples present transcriptomic profiles closer to each other than those of N1 samples (Data not shown). Further differential expression analysis among the three groups revealed 1886, 2998 and 592 differentially expressed genes with more than twofold change (adjusted (BH) P-value <0.05) between N1 and M1, N1 and M4, and M1 and M4 respectively, including 215 differentially expressed genes between the three groups (Data not shown). Thus the RNA sequencing results demonstrated the transcriptomic heterogeneity of nTregs.

B—Parental Maturation of nTreg Subsets.

The RNA sequencing supervised analysis confirms that each nTreg subset tested represents a maturation stage in nTreg life, even though, in resting nTreg cells, expression levels of mRNA and corresponding protein do not systematically parallel. The analysis focused on the mRNA expression of markers characterizing each a different phase of a T cell life. N1 to M1 to M4 maturation is reflected in mRNA expression levels of markers corresponding to cell 1) activation, 2) proliferation, 3) functional regulatory differentiation and 4) senescence (Table 1). Interestingly in this table of 40 relevant markers 1) KI-67 associated with cell cycle phases G1, S, G2, M but blocked in phase GO has been included in the activation but not in the IL-2 dependent proliferation phase, given the nTreg intrinsic inability to produce IL-2 required for proliferation. 2) Due to the low number of tested individuals (3 per subset), a trend but not a significant difference in the mRNA expression level of markers was most often observed. However individuals of each subset most often exhibited the same mRNA maturation profile, though each at a variable magnitude (Table 1).

IV—Medical Implications

1-NTreg CD39/CD26 Profile Provides a Novel Blood Biomarker for Monitoring Chronic Inflammatory Diseases and Post Irradiation Leukemias.

To investigate alteration of blood FOXP3 subpopulations in chronic inflammatory diseases, cryopreserved PBMCs of 12 untreated dermatomyositis (DM), 18 rheumatoid arthritis (RhA) both treated with immunosuppressive agents (representing auto-immune diseases associated with auto-antibodies and T cell activation) and 10 relapsing acute myeloid leukemia (AML) after hematopoietic stem cell transplantation (HSCT) were compared to 20 healthy adults (Table 2). The data are summarized in FIG. 4.

Auto-immunity: Whereas there was no difference in the CD4/CD8 ratio and in the frequency of CD4⁺ T cells and FOXP3⁺ cells within CD4⁺ T cells (data not shown), we observed great changes within the FOXP3⁺ population. In the FOXP3⁺ population, analysis of the flow cytometry profile in auto-immunity revealed a decrease of CD25⁺/25⁻ ratio (FIG. 4A1) and significant increase of CD127⁺/CD127⁻ ratio (FIG. 4A2). The same tendency was observed in SLE. FOXP3 MFI within subpopulations was higher in CD127⁻CD25⁻CD45RA⁻ compared to CD127⁻CD25⁻CD45RA⁺ and CD127⁺ (Data not shown) suggesting the accumulation of both activated CD127⁺ T helper cells expressing low levels of FOXP3 (outbound black disk, Data not shown) and nTregs loosing CD25⁺, as a marker of intense activation, expressing high levels of FOXP3 (outbound disk, Data not shown), consistent with previous findings (30). We next questioned whether the CD39/CD26 subset distribution of blood nTreg represents a novel pathogenic biomarker to monitor patients suffering from chronic inflammatory diseases. It revealed an elevation of the M4/M1 ratio (FIG. 4C1-2) in DM both in the CD25⁺ nTreg and the CD25⁻nTreg variant subsets in relation to an accumulation of the M4 terminal differentiated nTreg population. RhA was associated with marked increase of the N2 subpopulation above in healthy controls.

Moreover, abnormalities observed at diagnosis during DM reversed at least partially but significantly after treatment arguing for the use of CD39/CD26 subset distribution of blood nTreg as a biomarker of treatment response.

Acute myeloid leukemia: We observed a significant decrease of CD4+ T cells, as well as a decrease of the CD4⁺/CD8⁺ ratio and of CD45RA⁺ cells both in Tconv and nTregs (Data not shown) after HSCT as described (31). Patients suffering from AML after HSCT exhibited a highly decreased CD25⁺/CD25⁻ ratio (FIG. 4A1), indicating an elevation of a CD25⁻ abnormal variant. Concerning the CD39/CD26 subset distribution, the M4/M1 was highly elevated (FIG. 4C2) and the nTreg CD45RA⁺ distribution was skewed towards an accumulation of the N2, N3, N4 subsets (FIG. 4B1-2) and a decrease of the N1 as shown by the N4/N1 ratio (FIG. 4C1-2). The blood biomarkers study illustrates that within the blood FOXP3 T cell population, chronic inflammatory diseases are associated with an abnormal accumulation of FOXP3⁺CD25⁻ cells and FOXP3⁺CD127⁺ cells. Also the CD39/CD26 profile of FOXP3 memory cells skewed to a high expression of CD39⁺ marker (mainly subset M4), in DM and AML. AML and RhA were, moreover, associated with elevation of respectively both N1, N2 and N3 and N2 populations. Moreover, interestingly both high M4/M1 and N4/N1 ratio were associated with mortality during AML in relapse after HSCT (FIG. 4D1-2).

FIG. 5 depicts the evolution of Treg subpopulations after treatment during dermatomyositis. In particular, FIG. 5A shows the CD25+/CD25− frequency ratio among CD4⁺FOXP3⁺ T cells and FIG. 5B depict the frequency ratio of N4/N1 and M4/M1 among nTreg CD45RA⁺ and nTreg CD45RA⁻ respectively, defined by FOXP3⁺CD127⁻CD25⁺.

Discussion:

Although there have been over 50,000 publications on nTregs since their discovery in 1994 (1), our basic knowledge of these cells is still ill-defined and even confusing. Given their medical implication in autoimmune diseases (32) and their administration by adoptive transfer to treat these diseases or GVHD (24, 33), it is urgent that we obtain better understanding of these cells. The present study attempts to clarify some critical aspects on nTregs. The first question high lightened concerns as to which subsets of regulatory T cells belong to the nTregs lineage. These cells initially defined by their CD4⁺CD25⁺ phenotype (1), their natural thymic developmental origin, their necessary but not sufficient FOXP3 transcript expression, were further structurally well identified at a resting stage in human PBMCs, by their demethylated FOXP3-TSDR region (26), and lack of IL-2 production (27). These typic nTregs are distinguished from activated memory T cells originating from different CD4+ or CD8+ subtypes, which, under a tolerogenic microenvironment, transdifferentiate to express the regulatory FOXP3 transcript (17).

We further found in this study that human blood FOXP3 nTregs exhibit a CD39/CD26− based heterogeneous phenotypic profile comprised of 5 major subsets (FIG. 1). These subsets represent different stages of FOXP3 Treg maturation, as revealed by in vitro experiments carried out on separate subsets under different culture conditions critically including IL 2 and/or PGE2 and/or TGF β (FIG. 2). The supervised analysis of mRNA expression levels of markers implicated in the different phases of nTreg life (cell activation, proliferation, functional regulatory differentiation and senescence) confirm the subsets parental maturation (FIGS. 3A and 3B).

Most importantly, the present basic studies have relevant medical implications both for clinical diagnostics and therapy. At the clinical level, the finding that the CD39/CD26 profile was stable in healthy individuals over time but variable inter-individually prompted us to evaluate whether this profile represents a novel biomarker to be used for monitoring nTreg dysfunction in chronic inflammatory diseases. Initial phenotypic analysis carried out on PBMCs from AI patients suffering from DM, PAR and SLE as well as from AML patients after HSCT, not only showed disease-specific CD39/CD26 profiles but also abnormal expression of minor nTreg subsets including CD25-M4 variant, N2-4 subsets and FOXP3⁺ 127⁺ expressing M4 as illustrated in FIG. 4.

### REFERENCES

Throughout this application, various references describe the state of the art to which this invention pertains. The disclosures of these references are hereby incorporated by reference into the present disclosure.

1. Sakaguchi S, Sakaguchi N, Asano M, Itoh M, Toda M (1995) Immunologic Self-Tolerance Maintained by Activated T Cells Expressing 11-2 Receptor a-Chains (CD25). Breakdown of a single mechanism of self-tolerance causes various autoimmune diseases. J Immunol 155(3):1152-1164.

2. Sakaguchi S (2005) Naturally arising Foxp3-expressing CD25+ CD4+ regulatory T cells in immunological tolerance to self and non-self. Nat Immunol 6(4):345-352.

3. Gershon R K, Cohen P, Hencin R, Liebhaber S A (1972) Suppressor T cells. J Immunol 108(3):586-90.

4. Eardley D D, et al. (1978) Immunoregulatory circuits among T-cell sets. I. T-helper cells induce other T-cell sets to exert feedback inhibition. J Exp Med 147(4):1106-1115.

5. Groux H, et al. (1997) A CD4+ T-cell subset inhibits antigen-specific T-cell responses and prevents colitis. Nature 389(6652):737-742.

6. Vignali D A A, Collison L W, Workman C J (2008) How regulatory T cells work. Nat Rev Immunol 8(7):523-532.

7. Maizels R M, Smith K A (2011) Regulatory T Cells in Infection. Adv Immunol 112:73-136.

8. Takeuchi Y, Nishikawa H (2016) Roles of regulatory T cells in cancer immunity. Int Immunol 28(8):401-409.

9. Noval Rivas M, Chatila T A (2016) Regulatory T cells in allergic diseases. J Allergy Clin Immunol 138(3):639-652.

10. Beres A J, Drobyski W R (2013) The role of regulatory T cells in the biology of graft versus host disease. Front Immunol 4(June):163.

11. O'Garra A, Vieira P L, Vieira P, Goldfeld A E (2004) IL-10-producing and naturally occurring CD4+ Tregs: limiting collateral damage. J Clin Invest 114(10):1372-1378.

12. Maynard C L, Weaver C T (2008) Diversity in the contribution of interleukin-10 to T-cell-mediated immune regulation. Immunol Rev 226(1):219-233.

13. Le Buanec H, et al. (2011) IFN- and CD46 stimulation are associated with active lupus and skew natural T regulatory cell differentiation to type 1 regulatory T (Tr1) cells. Proc Natl Acad Sci 108(47):18995-19000.

14. Sarantopoulos S, Lu L, Cantor H (2004) Qa-1 restriction of CD8+ suppressor T cells. J Clin Invest 114(9):1218-1221.

15. Kim H-J, Lang P A, Cantor H (2013) CD8+ Treg—From Mouse To Man. Blood 122(21):3474.

16. Hori S, Nomura T, Sakaguchi S (2017) Control of regulatory T cell development by the transcription factor Foxp3. J Immunol 198(3):981-985.

17. Wang J, Ioan-Facsinay A, van der Voort E I H, Huizinga T W J, Toes R E M (2007) Transient expression of FOXP3 in human activated nonregulatory CD4+ T cells. Eur J Immunol 37(1):129-138.

18. Kmieciak M, et al. (2009) Human T cells express CD25 and Foxp3 upon activation and exhibit effector/memory phenotypes without any regulatory/suppressor function. J Transl Med 7(1):89.

19. Ayyoub M, et al. (2009) Human memory FOXP3+ Tregs secrete IL-17 ex vivo and constitutively express the TH17 lineage-specific transcription factor ROR t. Proc Natl Acad Sci 106(21):8635-8640.

20. Beriou G, et al. (2009) IL-17-producing human peripheral regulatory T cells retain suppressive function. Blood 113(18):4240-4249.

21. Chen W, et al. (2003) Conversion of Peripheral CD4 + CD25—Naive T Cells to CD4 + CD25 + Regulatory T Cells by TGF-β Induction of Transcription Factor Foxp3. J Exp Med 198(12):1875-1886.

22. Zheng S G, Wang J, Wang P, Gray J D, Horwitz D A (2007) IL-2 Is Essential for TGF- to Convert Naive CD4+CD25− Cells to CD25+Foxp3+ Regulatory T Cells and for Expansion of These Cells. J Immunol 178(4):2018-2027.

23. Huang S, Apasov S, Koshiba M, Sitkovsky M (1997) Role of Ata extracellular adenosine receptor-mediated signaling in adenosine-mediated inhibition of T-cell activation and expansion. Blood 90(4):1600-1610.

24. Gliwiński M, Iwaszkiewicz-Grześ D, Trzonkowski P (2017) Cell-Based Therapies with T Regulatory Cells. BioDrugs 31(4):335-347.

25. Thompson C B, et al. (1989) CD28 activation pathway regulates the production of multiple T-cell-derived lymphokines/cytokines. Proc Natl Acad Sci 86(4):1333-1337.

26. Baron U, et al. (2007) DNA demethylation in the humanFOXP3 locus discriminates regulatory T cells from activated FOXP3+ conventional T cells. Eur J Immunol 37(9):2378-2389.

27. Jonuleit H, et al. (2001) Identification and functional characterization of human CD4(+)CD25(+) T cells with regulatory properties isolated from peripheral blood. J Exp Med 193(11):1285-94.

28. Miyara M, et al. (2015) Sialyl Lewis x (CD15s) identifies highly differentiated and most suppressive FOXP3high regulatory T cells in humans. Proc Natl Acad Sci U S A 112(23):7225-30.

35.

29. Sallusto F, Geginat J, Lanzavecchia A (2004) Central Memory and Effector Memory T Cell Subsets: Function, Generation, and Maintenance. Annu Rev Immunol 22(1):745-763.

30. Ferreira R C, et al. (2017) Cells with Treg-specific FOXP3 demethylation but low CD25 are prevalent in autoimmunity. J Autoimmun 84:75-86.

31. Ogonek J, et al. (2016) Immune reconstitution after allogeneic hematopoietic stem cell transplantation. Front Immunol 7(November):1-15.

32. Paust S, Cantor H (2005) Regulatory T cells and autoimmune disease. Immunol Rev 204(1):195-207.

33. Trzonkowski P, et al. (2009) First-in-man clinical results of the treatment of patients with graft versus host disease with human ex vivo expanded CD4+CD25+CD127− T regulatory cells. Clin Immunol 133(1):22-26.

1. A population of Foxp3+ T cells selected from the group consisting of:
the population of Foxp3+ T cells having the following phenotype: CD45RA+CD26+CD39−,
the population of Foxp3+ T cells having the following phenotype: CD45RA+CD26−CD39−,
the population of Foxp3+ T cells having the following phenotype: CD45RA+CD26+CD39+,
the population of Foxp3+ T cells having the following phenotype: CD45RA+CD26−CD39+, and
the population of Foxp3+ T cells having the following phenotype: CD45RA−CD26+CD39−,
the population of Foxp3+ T cells having the following phenotype: CD45RA−CD26−CD39−,
the population of Foxp3+ T cells (M3) having the following phenotype: CD45RA-CD26+CD39+, and
the population of Foxp3+ T cells having the following phenotype: CD45RA−CD26−CD39+.

2. The population of Foxp3+ T cells of claim 1 which is CD4⁺ or CD8⁺.

3. The population of Foxp3+ T cells of claim 1 which is CD25⁺ or CD25⁻.

4. The population of Foxp3+ T cells of claim 1 which is selected from the group consisting of:
the population of CD4⁺ Foxp3⁺ regulatory T cells (N1) having the following phenotype: CD25⁺CD45RA⁺CD26⁺CD39⁻,
the population of CD4⁺ Foxp3⁺ regulatory T cells (N2) having the following phenotype: CD25⁺CD45RA⁺CD26⁻CD39⁻,
the population of CD4⁺ Foxp3⁺ regulatory T cells (N3) having the following phenotype: CD25⁺CD45RA⁺CD26⁺CD39⁺,
the population of CD4⁺ Foxp3⁺ regulatory T cells (N4) having the following phenotype: CD25⁺CD45RA⁺CD26⁻CD39⁺,
the population of CD4⁺ Foxp3⁺ regulatory T cells (M1) having the following phenotype: CD25⁺CD45RA⁻CD26⁺CD39⁻,
the population of CD4⁺ Foxp3⁺ regulatory T cells (M2) having the following phenotype: CD25⁺CD45RA⁻CD26⁻CD39⁻,
the population of CD4⁺ Foxp3⁺ regulatory T cells (M3) having the following phenotype: CD25⁺CD45RA⁻CD26⁺CD39⁺, and
the population of CD4⁺ Foxp3⁺ regulatory T cells (M4) having the following phenotype: CD25⁺CD45RA⁻CD26⁻CD39⁺.

5. The population of Foxp3+ T cells of claim 1 which is selected from the group consisting of:
the population of CD4⁺ Foxp3⁺ regulatory T cells (N1′) having the following phenotype: CD25⁻CD45RA⁺CD26⁺CD39⁻,
the population of CD4⁺ Foxp3⁺ regulatory T cells (N2′) having the following phenotype: CD25⁻CD45RA⁺CD26⁻CD39⁻,
the population of CD4⁺ Foxp3⁺ regulatory T cells (N3′) having the following phenotype: CD25⁻CD45RA⁺CD26⁺CD39⁺,
the population of CD4⁺ Foxp3⁺ regulatory T cells (N4′) having the following phenotype: CD25⁻CD45RA⁺CD26⁻CD39⁺,
the population of CD4⁺ Foxp3⁺ regulatory T cells (M1′) having the following phenotype: CD25⁻CD45RA⁻CD26⁺CD39⁻,
the population of CD4⁺ Foxp3⁺ regulatory T cells (M2′) having the following phenotype: CD25⁻CD45RA⁻CD26⁻CD39⁻,
the population of CD4⁺ Foxp3⁺ regulatory T cells having (M3′) the following phenotype: CD25⁻CD45RA⁻CD26⁺CD39⁺, and
the population of CD4⁺ Foxp3⁺ regulatory T cells having (M4′) the following phenotype: CD25⁻CD45RA⁻CD26⁻CD39⁺.

6. (canceled)

7. A method of assessing the maturation stage of a population of Foxp3+ T cells comprising i) detecting the expression of the phenotypic markers CD45RA, CD25, CD26 and CD39 in said population of Foxp3+ T cells and ii) determining a category of maturation stage to which said population of Foxp3+ T cells belongs.

8. (canceled)

9. A method of determining whether a subject suffers from an impaired immune response and/or immunosenescence comprising i) quantifying the amount of at least one population of Foxp3+ T cells according to claim 1 in a sample obtained from the subject, and ii) comparing the amount quantified at step i) with a predetermined reference value wherein detecting a differential between said amount and said predetermined reference value indicates whether the subject suffers from an impaired immune response and/or immunosenescence.

10. A method of determining whether a subject has or is at risk of having a disease that is an autoimmune inflammatory disease, an infectious disease or a cancer and treating the disease comprising i) quantifying the amount of at least one population of Foxp3+ T cells according to claim 1 in a sample obtained from the subject, and ii) treating the disease when there is a difference between the amount quantified in step i) and a corresponding reference value based on healthy controls.

11. A method of determining whether a patient suffering from an autoimmune inflammatory disease, an infectious disease or a cancer will achieve a therapeutic response with a treatment comprising i) quantifying the amount of at least one population of Foxp3+ T cells according to claim 1 in a sample obtained from the patient and ii) and comparing the amount quantified at step i) with a predetermined reference value wherein detecting a differential between said amount and said predetermined reference value indicates whether the patient achieves or does not achieve a therapeutic response with the treatment.

12. A method of determining whether a patient suffering from an autoimmune inflammatory disease, an infectious disease or a cancer is at risk of relapse comprising i) quantifying the amount of at least one population of Foxp3+ T cells according to claim 1 in a sample obtained from the patient, and ii) comparing the amount quantified at step i) with a predetermined reference value wherein detecting a differential between said amount and said predetermined reference value indicates whether the patient is or is not at risk of relapse.

13. A method for predicting the survival time of a subject suffering from a cancer comprising i) quantifying the amount of at least one population of Foxp3+ T cells according to claim 1 in a sample obtained from the subject, and ii) comparing the amount quantified at step i) with a predetermined reference value wherein detecting a differential between said amount and said predetermined reference value indicates whether the patient will have a short or long survival time.

14. The method according to claim 9, wherein at least 2, 3, 4, 5, 6, 7, 8, 9, 10, 11, 12, 13, 14, 15 or 16 populations of Foxp3+ T cells are quantified.

15. The method according to claim 9, wherein the quantification is absolute or relative.

16. The method according to claim 9, wherein the quantification is relative to a population of Foxp3+ T according to claim 1.

17. The method according to claim 9, wherein a ratio between at least 2 populations of cells is calculated and compared to a predetermined reference value, and wherein detecting a differential between said amount and said predetermined reference value indicates respectively:
whether the subject suffers from an impaired immune response and/or immunosenescence, or
whether the subject has or is at risk of having an autoimmune inflammatory disease, an infectious disease or a cancer, or
whether the patient will achieve or will not achieve a therapeutic response treatment with a CD39 inhibitor, or
whether the patient is or is not at risk of relapse, or
whether the subject has a short or long survival time.

18. The method of claim 17 wherein the ratio between the population (M4) and (M1) is determined, and wherein the higher the M4/M1 ratio, the higher is the probability that the subject has or is at risk of having an autoimmune inflammatory disease.

19. The method of claim 17 wherein the ratio between the population (N4) and (N1) is determined, and wherein the higher the N4/N1 ratio, the higher is the probability that the subject has or is at risk of having a cancer.

20. A method of determining whether a patient suffering from cancer is eligible for treatment with a CD39 inhibitor and if so, treating the patient with the CD39 inhibitor comprising i) quantifying the amount of a population of CD39+ cells selected from the group consisting of selected from the group consisting of:
the population of Foxp3+ T cells having the following phenotype: CD45RA+CD26+CD39−,
the population of Foxp3+ T cells having the following phenotype: CD45RA+CD26−CD39−,
the population of Foxp3+ T cells having the following phenotype: CD45RA+CD26+CD39+,
the population of Foxp3+ T cells having the following phenotype: CD45RA+CD26−CD39+, and
the population of Foxp3+ T cells having the following phenotype: CD45RA−CD26+CD39−,
the population of Foxp3+ T cells having the following phenotype: CD45RA−CD26−CD39−,
the population of Foxp3+ T cells (M3) having the following phenotype: CD45RA−CD26+CD39+, and
the population of Foxp3+ T cells having the following phenotype: CD45RA−CD26−CD39+, and ii) treating the patient with the CD39 inhibitor when a difference between the amount and a predetermined reference value is detected.

21. A kit comprising a panel of antibodies specific for CD3, CD4, CD8, Foxp3, CD25, CD45RA, CD26 and CD39.

22. The method of claim 7, wherein the category is N1, N2, N3, N4, M1, M2, M3 or M4.

23. The method of claim 10, wherein
the disease is cancer,
the at least one population of Foxp3+ T cells includes the N1, N2, N3 and N4 naïve populations of Foxp3+ T cells, and
the step of treating is performed when an increase of the N2, N3 and N4 naive populations and/or a decrease of the N1 naive population is detected, compared to the corresponding reference value.

24. The method of claim 10, wherein
the disease is an autoimmune inflammatory disease,
the at least one population of Foxp3+ T cells includes the CD25+, CD25−, CD127+ and CD127− populations of Foxp3+ T cells, and
the step of treating is performed when a ratio of CD25+/CD25− cells is decreased and a ratio of CD127+/CD127− is increased, compared to the corresponding reference value.


SEQUENCE LISTING (6 sequences)

SEQ ID NO: 1 - protein, 431 aa, Homo sapiens
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 2 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 3 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 4 - protein, 510 aa, Homo sapiens
MEDTKESNVKTFCSKNILAILGFSSIIAVIALLAVGLTQNKALPENVKYGIVLDAGSSHTSLYIYKWPAEKENDTGVVHQVEECRVKGPGISKFVQKVNEIGIYLTDCMERAREVIPRSQHQETPVYLGATAGMRLLRMESEELADRVLDVVERSLSNYPFDFQGARIITGQEEGAYGWITINYLLGKFSQKTRWFSIVPYETNNQETFGALDLGGASTQVTFVPQNQTIESPDNALQFRLYGKDYNVYTHSFLCYGKDQALWQKLAKDIQVASNEILRDPCFHPGYKKVVNVSDLYKTPCTKRFEMTLPFQQFEIQGIGNYQQCHQSILELFNTSYCPYSQCAFNGIFLPPLQGDFGAFSAFYFVMKFLNLTSEKVSQEKVTEMMKKFCAQPWEEIKTSYAGVKEKYLSEYCFSGTYILSLLLQGYHFTADSWEHIHFIGKIQGSDAGWTLGYMLNLTNMIPAEQPLSTPLSHSTYVFLMVLFSLVLFTVAIIGLLIFHKPSYFWKDMV

SEQ ID NO: 5 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 6 - protein, 459 aa, Homo sapiens [sequence omitted]